(19) United States
(12) Patent Application Publication
(10) Pub. No.: US 2009/0018507 A1
(43) Pub. Date: Jan. 15, 2009

(54) SPINAL ACCESS SYSTEM AND METHOD

(75) Inventors: Gregory P. Schmitz, Mountain View, CA (US); Jeffery L. Bleich, Mountain View, CA (US); Roy Leguidleguid, Mountain View, CA (US); Eric C. Miller, Mountain View, CA (US); Gregory B. Arcenio, Mountain View, CA (US); Michael P. Wallace, Mountain View, CA (US)

Correspondence Address: SHAY GLENN LLP, 2755 CAMPUS DRIVE, SUITE 210, SAN MATEO, CA, 94403, US

(73) Assignee: Baxano, Inc., Mountain View, CA (US)

(21) Appl. No.: 12/170,392

(22) Filed: Jul. 9, 2008

Related U.S. Application Data
(60) Provisional application No. 60/948,664, filed on Jul. 9, 2007.
(60) Provisional application No. 61/048,448, filed on Apr. 28, 2008.

(51) Int. Cl. A61M 5/31; A61B 5/055; A61M 5/32

(52) U.S. Cl. 604/164.03; 604/164.13; 604/164.06; 600/424; 604/164.04

(57) ABSTRACT

Described herein are devices and systems for accessing a spine and particularly the epidural region of the spine and methods of using these systems and devices to access the spine or regions of the spine. In particular, cannulas that may be anchored to the ligamentum flavum or the periosteum are described. Ligamentum flavum access tools are also described. These tools may be used with (or without) an anchoring cannula to penetrate the ligamentum flavum and provide access to the epidural space without risk of injury to other structures within the epidural space. The devices, methods and systems described herein are particularly useful in minimally invasive surgical (MIS) uses. The devices, methods and systems described herein may be used for performing spinal decompressions and other spinal procedures.

### CROSS REFERENCE TO RELATED APPLICATIONS

This application claims priority to U.S. Provisional Patent Applications No. 60/948,664, filed Jul. 9, 2007 (titled “SPINAL ACCESS SYSTEM AND METHOD”) and 61/048,448, filed Apr. 28, 2008 (titled “EPIDURAL ACCESS TOOLS AND METHODS”).

### INCORPORATION BY REFERENCE

All publications and patent applications mentioned in this specification are herein incorporated by reference in their entirety as if each individual publication or patent application was specifically and individually indicated to be incorporated by reference.

### FIELD OF THE INVENTION

The present invention relates generally to medical/surgical devices and methods. More specifically, the present invention relates to a spinal access system and method.

### BACKGROUND OF THE INVENTION

In recent years, less invasive (or “minimally invasive”) surgical techniques have become increasingly more popular, as physicians, patients and medical device innovators have sought to reduce the trauma, recovery time and side effects often associated with conventional surgery. Developing less invasive surgical methods and devices, however, poses many challenges. For example, less invasive techniques typically involve working in a smaller operating field, working with smaller devices, and trying to operate with reduced or even no direct visualization of the structures being treated. These challenges are often compounded when target tissues of a given procedure reside very close to one or more vital, non-target tissues.

One area of surgery which would likely benefit from the development of less invasive techniques is the treatment of spinal stenosis. Spinal stenosis occurs when nerve tissue and/or the blood vessels supplying nerve tissue in the spine become impinged by one or more structures pressing against them, causing symptoms. The most common form of spinal stenosis occurs in the lower (or lumbar) spine and can cause severe pain, numbness and/or loss of function in the lower back and/or one or both lower limb.

FIG. 1 is a top view of a vertebra with the cauda equina (the bundle of nerves that extends from the base of the spinal cord) shown in cross section and two nerve roots branching from the cauda equina to exit the central spinal canal and extend through intervertebral foramina (or “neural foramina”—singular “foramen”) on either side of the vertebra. Spinal stenosis can occur when the spinal cord, cauda equina and/or nerve root(s) are impinged by one or more tissues in the spine, such as buckled or thickened ligamentum flavum, hypertrophied facet joint (shown as superior articular processes shown in FIG. 1), osteophytes (or “bone spurs”) on vertebrae, spondylolisthesis (sliding of one vertebra relative to an adjacent vertebra), facet joint synovial cysts, and/or collapse, bulging or herniation of an intervertebral disc. Impingement of neural and/or neurovascular tissue in the spine by one or more of these tissues may cause pain, numbness and/or loss of strength or mobility in one or both of a patient's lower limbs and/or of the patient's back.

In the United States, spinal stenosis occurs with an incidence of between 4% and 6% of adults aged 50 and older and is the most frequent reason cited for back surgery in patients aged 60 and older. Patients suffering from spinal stenosis are typically first treated with conservative approaches such as exercise therapy, analgesics, anti-inflammatory medications, and epidural steroid injections. When these conservative treatment options fail and symptoms are severe, as is frequently the case, surgery may be required to remove impinging tissue and decompress the impinged nerve tissue.

Lumbar spinal stenosis surgery involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or laminotomy). In addition, the surgery often includes partial or complete facetectomy (removal of all or part of one or more facet joints), to remove impinging ligamentum flavum or bone tissue. Spinal stenosis surgery is performed under general anesthesia, and patients are usually admitted to the hospital for five to seven days after surgery, with full recovery from surgery requiring between six weeks and three months. Many patients need extended therapy at a rehabilitation facility to regain enough mobility to live independently.

Removal of vertebral bone, as occurs in laminectomy and facetectomy, often leaves the affected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

A number of devices, systems and methods for less invasive treatment of spinal stenosis have been described by the assignee of the present invention. For example, various embodiments of such devices, systems and methods are described in U.S. patent application Ser. Nos.: 11/250,332, entitled “DEVICES AND METHODS FOR SELECTIVE SURGICAL REMOVAL OF TISSUE,” and filed Oct. 15, 2005; 11/375,265, entitled “METHOD AND APPARATUS FOR TISSUE MODIFICATION,” and filed Mar. 13, 2006; 11/251,155, entitled “DEVICES AND METHODS FOR TISSUE ACCESS” and filed Oct. 15, 2005; 11/952,934, entitled “TISSUE REMOVAL DEVICES AND METHODS” and filed Dec. 7, 2007; and 11/535,000, entitled “TISSUE CUTTING DEVICES AND METHODS,” and filed Sep. 25, 2006, all of which applications are hereby incorporated fully by reference herein.

One challenge in treating spinal stenosis using minimally invasive tools is accessing the small, confined spaces of the spine to address impinging tissues. In conventional surgical approaches, as mentioned above, access is generally gained by performing a laminotomy or laminectomy in the vertebrae. Even in these open surgical approaches, it is often difficult or impossible to see or reach an intervertebral foramen where tissue may be impinging a nerve root. In less invasive procedures, accessing an intervertebral foramen is usually even more difficult.

A number of devices, systems and methods for accessing target tissue in the spine and identifying neural tissue have been identified. For example, tissue access is addressed in U.S. patent application Ser. Nos.: 11/251,205, entitled “DEVICES AND METHODS FOR TISSUE ACCESS,” and filed Oct. 15, 2005; 11/457,416, entitled “SPINAL ACCESS AND NEURAL LOCALIZATION,” and filed Jul. 13, 2006; and 11/468,247, entitled “TISSUE ACCESS GUIDEWIRE SYSTEM AND METHOD,” and filed Aug. 29, 2006, all of which applications are hereby incorporated fully by reference herein. Assignee of the present invention has described a number of devices, systems and methods for removing or otherwise treating target tissue in the spine in U.S. patent application Ser. Nos.: 11/251,165, entitled “DEVICES AND METHODS FOR TISSUE MODIFICATION,” and filed Oct. 15, 2005; 11/375,265, entitled “METHODS AND APPARATUS FOR TISSUE MODIFICATION,” and filed Mar. 13,2006; 11/535,000, entitled “TISSUE CUTTING DEVICES AND METHODS,” and filed Sep. 5, 2006; and 11/687,558, entitled “Flexible TISSUE REMOVAL DEVICES AND METHODS,” and filed Mar. 16, 2007; all of which applications are hereby incorporated fully by reference herein. Although the inventions described in these applications solve many of the challenges associated with minimally invasive or less invasive spinal access, further innovations and improvements are always desirable.

Therefore, it would be desirable to have improved systems and methods for accessing a spine. Ideally, such systems and methods would work in a minimally invasive, less invasive and/or percutaneous access settings, without requiring large incisions, laminotomies, laminectomies, or direct visualization of the site to be accessed. In some cases, it may be ideal to provide access to one or more intervertebral foramina of the spine, while it may also or alternatively be desirable to provide access to the central spinal canal. At least some of these objectives will be met by the present invention.

### SUMMARY OF THE INVENTION

Described herein are systems for accessing a spine and particularly the epidural region of the spine, devices for accessing the spine, and methods of using these systems and devices to access the spine or regions of the spine. In particular, cannulas that may be anchored to the ligamentum flavum or the periosteum are described. Other access methods and associated tools for achieving safe and reliable spinal (e.g., epidural) access are also described. In particular, ligamentum flavum access tools are described. These tools may be used with (or without) an anchoring cannula to penetrate the ligamentum flavum and provide access to the epidural space without risk of injury to other structures within the epidural space. The devices, methods and systems described herein are particularly useful in minimally invasive surgical (MIS) uses. For example, these tools and methods may be useful in percutaneous procedures. Any of these tools may also be used in an open surgical setting as well. The devices, methods and systems described herein may be used for performing spinal decompressions and other spinal procedures.

For example, anchoring cannula, systems including them, and procedures using them are described. Although a cannula may be anchored either to the patient or to a structure outside of the patient, for many of the methods described herein it may be particularly helpful to provide a cannula that is distally anchorable to a spinal structure such as the ligamentum flavum or the periosteum of the spine. For example, described herein are methods for accessing a spine of a patient may involve advancing a cannula into the patient to contact a distal end of the cannula with spinal tissue including at least one of ligamentum flavum or vertebral periosteum, removeably attaching the distal end of the cannula to at least one of the ligamentum flavum or the periosteum and/or bone, advancing a curved, at least partially flexible, cannulated guide member through the cannula and through at least one of the ligamentum flavum or vertebral periosteum to position a distal portion of the guide member in the epidural space of the spine, such that when the distal portion exits the cannula it assumes a preformed curved shape, and advancing the distal portion of the guide member at least partway into an intervertebral foramen of the spine.

In some embodiments, the cannula may be advanced along with an epidural needle, with the cannula disposed over the needle as a sheath, and the method further involve removing the needle before advancing the guide member through the cannula. For example, in one embodiment, removing the needle may involve ejecting the epidural needle proximally to remove a tip of the needle from the epidural space and sliding the needle proximally out of the cannula. In an alternative embodiment, the cannula may be advanced along with a blunt stylet, with the cannula disposed over the stylet as a sheath, and the method may further involve removing the stylet before advancing the guide member through the cannula. In some embodiments, rather than (or in addition to) a needle, a ligamentum flavum access tool may be used to penetrate the ligamentum flavum. Ligamentum flavum access tools are described in greater detail blow.

In some embodiments, attaching the distal end of the cannula to the tissue may involve turning the cannula about its longitudinal axis in a first direction to couple one or more barbs disposed on its distal end with the tissue. Such a method may farther involve turning the cannula about its longitudinal axis in a second direction, opposite the first direction, to release the cannula from the tissue, after advancing the guide member into the intervertebral foramen. In some embodiments, the method may also include, before advancing the guide member, advancing a rigid, blunt, cannulated probe through the cannula to position a distal end of the probe in the epidural space, wherein the curved guide member is advanced through the rigid probe.

In one embodiment, the method may also include advancing a guidewire through the guide member to pass through the intervertebral foramen and out the patient's skin, releasing the cannula from the spinal tissue, and removing the cannula and the guide member from the patient, leaving the guidewire in place, extending into the patient, through the intervertebral foramen, and back out the patient. Such a method may also optionally include coupling a tissue removal device with the guidewire, advancing the tissue removal device at least partway into the intervertebral foramen, using the guidewire, and performing a tissue removal procedure in the patient's spine.

In one embodiment, the method may further involve transmitting stimulating current to at least one electrode disposed on the curved guide member to help determine a position of the guide member relative to nerve tissue. For example, transmitting the current may involve transmitting a first current to a first electrode disposed on an inner curvature surface of the guide member and transmitting a second current to a second electrode disposed on an outer curvature surface of the guide member. In some embodiments, the method may further include, before the transmitting step, advancing a sheath comprising at least one electrode over the guide member into the epidural space of the spine. In an alternative embodiment, the method may further involve, before advancing the guide member, advancing at least one additional cannula over the attached cannula, removeably attaching the additional cannula to the spinal tissue, removing the cannula from the tissue, and withdrawing the cannula through the additional cannula.

Also described herein are methods for advancing a guidewire through an intervertebral foramen of a spine of a patient may involve advancing a cannula into the patient to contact a distal end of the cannula with spinal tissue including at least one of ligamentum flavum or vertebral periosteum, removeably attaching the distal end of the cannula to at least one of the ligamentum flavum or the periosteum, advancing a curved, at least partially flexible, cannulated guide member through the cannula and through at least one of the ligamentum flavum or vertebral periosteum to position a distal portion of the guide member in the epidural space of the spine, such that when the distal portion exits the cannula it assumes a preformed curved shape, advancing the distal portion of the guide member at least partway into an intervertebral foramen of the spine, advancing a guidewire through the guide member to pass through the intervertebral foramen and out the patient's skin, releasing the cannula from the spinal tissue, and removing the cannula and the guide member from the patient, leaving the guidewire in place, extending into the patient, through the intervertebral foramen, and back out the patient.

Also described herein are methods for advancing a guidewire through an epidural space of a spine of a patient may involve advancing a cannula into the patient to contact a distal end of the cannula with spinal tissue including at least one of ligamentum flavum or vertebral periosteum, removeably attaching the distal end of the cannula to at least one of the ligamentum flavum or the periosteum, advancing a curved, at least partially flexible, cannulated guide member through the cannula and between first and second vertebrae to position a distal portion of the guide member in the epidural space of the spine, such that when the distal portion exits the cannula it assumes a preformed curved shape, advancing the distal portion of the guide member at least partway between the second vertebra and a third vertebra of the spine, advancing a guidewire through the guide member to pass between the second and third vertebrae and out the patient's skin, releasing the cannula from the spinal tissue, and removing the cannula and the guide member from the patient, leaving the guidewire in place, extending between the first and second vertebrae, through the epidural space, between the second and third vertebrae, and back out the patient.

In another variation, a method for accessing an intervertebral foramen of a spine of a patient may suitably include removeably attaching a distal end of a first tissue locking cannula to spinal tissue including at least one of ligamentum flavum or vertebral periosteum, passing at least a second tissue locking cannula over the first cannula, removeably attaching a distal end of the second cannula to the spinal tissue, removing the first cannula through the second cannula, advancing a probe through the second cannula to position a distal portion of the probe in an epidural space of the patient's spine, advancing a curved, at least partially flexible, cannulated guide member through the probe, such that when the distal portion exits the cannula it assumes a preformed curved shape, and advancing the distal portion of the guide member at least partway into an intervertebral foramen of the spine.

In some embodiments, the method may further include advancing a guidewire through the guide member to pass through the intervertebral foramen and out the patient's skin, removing the probe from the patient, releasing the second cannula from the spinal tissue, and removing the cannula from the patient, leaving the guidewire in place, extending into the patient, through the intervertebral foramen, and back out the patient. In some embodiments, the method may further include, before advancing the probe, passing at least a third tissue locking cannula over the second cannula, removeably attaching a distal end of the third cannula to the spinal tissue; and removing the second cannula through the third cannula. In one embodiment, the method may further include, before advancing the probe, passing at least a fourth tissue locking cannula over the third cannula, removeably attaching a distal end of the fourth cannula to the spinal tissue, and removing the third cannula through the fourth cannula.

Also described herein are systems for accessing a spine of a patient may include at least one tissue locking cannula having multiple barbs disposed at one end for removeably attaching to spinal tissue including at least one of ligamentum flavum or vertebral periosteum, at least one of a needle or a stylet slideably disposed in the cannula, and a curved, at least partially flexible, cannulated guide member slideably passable through the cannula and having a distal portion configured to change from a straight shape within the cannula to a curved shape upon exiting the cannula, wherein the distal portion has a radius of curvature configured to position the distal portion at least partway into an intervertebral foramen of the spine when advanced through the cannula.

Some embodiments may further include a rigid, cannulated probe slideably passable through the cannula, wherein the curved guide member slide ably passes through the probe. In some embodiments, the guide member may pass through an end aperture of the probe. In alternative embodiments, the guide member may pass through a side aperture of the probe. In some embodiments, the system may further include at least one guidewire for passing through the guide member. In some embodiments, the system may further include a syringe for attaching to a proximal portion of the epidural needle. In some embodiments, the system may further include a tissue removal device removeably couplable with the guidewire for passing into the patient to remove spinal tissue.

In some embodiments, the tissue locking cannula may have an outer diameter of between about 1 mm and about 20 mm. In one embodiment, the barbs of the cannula may face in one direction and attach to tissue by pressing the barbs against the tissue and turning the cannula along its longitudinal axis in a first direction. In one embodiment, the barbs may release from tissue by turning the cannula along its longitudinal axis in a second direction opposite the first direction. In some embodiments, the guide member may include a rounded, atraumatic distal tip. In some embodiments, the at least one tissue locking cannula may include multiple cannulas of different diameter, wherein a first cannula fits within a second cannula, and the second cannula fits within at least a third cannula.

Also described herein are systems for accessing a spine of a patient may include: multiple tissue locking cannulas, each cannula having a different diameter such that larger cannulas slide over smaller cannulas, and each cannula having multiple barbs disposed at one end for removeably attaching to spinal tissue including at least one of ligamentum flavum or vertebral periosteum; a cannulated probe passable through at least a largest diameter cannula of the multiple cannulas; and a curved, at least partially flexible, cannulated guide member slide ably passable through the probe and having a distal portion configured to change from a straight shape within the probe to a curved shape upon exiting the probe, wherein the distal portion has a radius of curvature configured to position the distal portion at least partway into an intervertebral foramen of the spine when advanced through the probe.

In some embodiments, the multiple cannulas may include between two and six cannulas. In some embodiments, the probe may comprise a rigid probe including an approximately straight shaft portion and a curved distal portion, wherein the curve has an angle of curvature configured to allow the distal portion to pass through at least a largest of the cannulas.

As mentioned above, the tissue locking (anchoring) spinal access systems described above, including the distally anchoring cannula, may be used with other access or spinal surgical tools. For example, any of the devices and systems described above may be used with one or more ligamentum flavum access tools. In general, a ligamentum flavum access system includes an outer cannula (which may be a distally anchoring cannula as described above), and an inner member that is controllably movable relative to the outer member. In some variations, an additional cannula is used, which fits within the outer cannula, and allows passage of the inner member. The system is typically configured to penetrate the ligamentum flavum and cut or expand an opening therethrough, so that a procedure may be performed on the spine. Any of these devices may also include one or more detectors for detecting when the system has penetrated the ligamentum flavum and into the epidural space. For example, the system may include a hole or opening near the distal end for detecting a loss of resistance once a portion of the system has penetrated the ligamentum flavum.

For example, described herein is a ligamentum flavum access tool device comprising an outer hypotube having a distal cutting edge and an inner member having an atraumatic tissue contacting region that is movable within the outer hypotube, and extends from the outer hypotube, wherein the inner member is configured to secure to a patient's ligamentum flavum. The device may also include a loss of resistance detector configured to determine when the inner member is within the epidural space.

In some variations, the inner member includes a vacuum port configured to provide a vacuum for securing the inner member to the ligamentum flavum. For example, the inner member may be an inner hypotube (e.g., cannula) that includes an opening for applying a vacuum to hold the ligamentum flavum securely. The outer hypotube (cannula) may have a sharpened edge, so that by moving the outer hypotube relative to the inner hypotube, a hole in the ligamentum flavum may be cut. In any of variations one or both of the inner and outer members (e.g. an outer hypotube including a sharpened edge) may be rotatable relative to the inner member, which may help with cutting of the ligamentum flavum.

In some variations, the devices include at least one support element extendable from the inner member when the inner member is within the epidural space. For example, the inner member may include one or more arms that extend from the distal region of the inner member after it has passed into the epidural space, so that these arms or other extendable elements may support the ligamentum flavum so that it can be cut. In some variations the support element(s) are arms made of Nitinol or other shape-memory or appropriately deformable material that may be extended from the inner member (e.g., substantially perpendicular to the long axis of the inner member.

In some variations, the atraumatic tissue contacting region of the inner member includes a distal head and a proximal neck that has a smaller diameter than the distal head, wherein the ligamentum flavum may be secured around the proximal neck after the distal head has penetrated the ligamentum flavum. For example, the inner member may have a “mushroom” shape that permits the tissue to be secured around the narrower neck region after this head portion penetrates the ligamentum flavum.

Any of these devices (tools) may also include a threaded region on an outer surface of the device that is configured to mate with a cannula so that the device may be controllably advanced within the cannula by rotation. Furthermore, the cannula may be an anchoring cannula that includes complimentary threads for advancing the tool by rotating.

In addition, the devices may also include an internal threaded region in communication with the inner atraumatic tissue contacting member so that it may be moved relative to the outer hypotube. For example, in some variations the inner and outer members may be drawn together to cut the ligamentum flavum.

Also described herein are ligamentum flavum access tool devices comprising an elongate body, a distal tip member comprising an atraumatic tissue contacting region configured as a leading head, a cutting surface that is located proximal to the distal tip member, and a loss of resistance detector, configured to determine when the distal tip member is within the epidural space. The cutting surface may be located on a proximal side of the leading head of the distal tip member. In other variations, the cutting surface is a cutting edge of a hypotube in which the distal tip member may axially move.

In some variations, the devices include at least one support element extendable from the distal tip member when the distal tip member is within the epidural space. The distal tip member may be axially movable relative to the cutting surface.

As mentioned above, the device may also include a threaded region on an outer surface of the device that is configured to mate with a cannula so that the device may be controllably advanced within the cannula by rotation, and/or an internal threaded region in communication with the distal tip member so that the distal tip member may be moved relative to the cutting surface.

Also described herein are ligamentum flavum access tool devices comprising a proximal hypotube having an expandable distal end, and a distal tip member comprising an atraumatic leading that is axially movable relative to the proximal hypotube, and a loss of resistance detector, configured to determine when the proximal hypotube is within the epidural space. The expandable distal end of the proximal hypotube may include a plurality of axial slits.

In some variations, the proximal hypotube is configured to be anchored in position within the ligamentum flavum.

Also described herein are systems for accessing a patient's spine. For example, a system may include a cannula configured to be anchored in contact with the ligamentum flavum, a ligamentum flavum access tool configured to be controllably advanced within the cannula, and a curved cannulated guide member passable through the cannula and having a distal portion configured to change from a straight shape within the cannula to a curved shape upon exiting the cannula, wherein the distal portion of the guide member has a radius of curvature configured to position the distal portion at least partway into an intervertebral foramen of the spine when advanced through the cannula. The ligamentum flavum access tool may include any of those described herein. For example, the ligamentum flavum access tool may include a proximal hypotube having a cutting edge, and a distal atraumatic tissue contacting region that is movable relative to the proximal hypotube. The ligamentum flavum access tool may include a threaded region that mates with a threaded portion of the cannula so that the ligamentum flavum access tool may be controllably advanced by rotation.

In some variations, the ligamentum flavum access tool further comprises a load of resistance detector.

The distal atraumatic tissue contacting region of the ligamentum flavum access tool may include a leading head having an atraumatic surface. In some variations, the distal atraumatic tissue contacting region of the ligamentum flavum access tool comprises a vacuum port configured to secure the ligamentum flavum to the distal atraumatic tissue contacting region.

In some variations, the distal atraumatic tissue contacting region of the ligamentum flavum access tool may include at least one support element extendable from the atraumatic tissue contacting region when the atraumatic tissue contacting region is within the epidural space.

Any of the systems for accessing the spine described herein may include any of the elements described above for performing a spinal procedure, particularly a spinal decompression procedure. For example, the system may also include a cannulated probe configured to allow the cannulated guide member to pass and further configured to pass through the cannula, and/or at least one guidewire configured to pass through the cannulated guide member.

The cannula included as part of the system may be a tissue locking cannula as described above, such as a cannula having a plurality of barbs disposed at or near the distal end for removeably anchoring the locking cannula in communication with the ligamentum flavum.

Also described herein are systems for accessing a patient's spine including a cannula configured to be anchored in contact with the ligamentum flavum, a ligamentum flavum access tool configured to be controllably advanced within the cannula, and a curved cannulated guide member passable through the cannula and having a distal portion configured to change from a straight shape within the cannula to a curved shape upon exiting the cannula, wherein the distal portion of the guide member has a radius of curvature configured to position the distal portion at least partway into an intervertebral foramen of the spine when advanced through the cannula. The ligamentum flavum access tool may include a proximal hypotube having an expandable distal end, and a distal tip member comprising an atraumatic leading that is movable relative to the proximal hypotube.

The distal tip member may be further configured to expand the expandable distal end of the proximal hypotube when the distal tip member is passed through the proximal hypotube.

Also described herein are systems for accessing a patient's spine, the system comprising a cannula configured to be anchored in contact with the ligamentum flavum, a ligamentum flavum access tool configured to be controllably advanced within the cannula, and a curved cannulated guide member passable through the cannula and having a distal portion configured to change from a straight shape within the cannula to a curved shape upon exiting the cannula, wherein the distal portion of the guide member has a radius of curvature configured to position the distal portion at least partway into an intervertebral foramen of the spine when advanced through the cannula. The ligamentum flavum access tool may comprise a proximal cutting surface, a distal tip member comprising an atraumatic tissue contacting region configured as a leading head, and a loss of resistance detector.

Methods of accessing the spine using any of the elements described above, such as the spinal access tool device, and systems including them, may be performed either percutaneously or in an open procedure. In particular any of these devices, tools or systems may be used as part of a procedure for accessing the epidural space of the spine.

For example, described herein are methods of accessing the spine of a patient comprising the steps of: anchoring the distal end of a cannula in contract with a patient's ligamentum flavum; advancing a ligamentum flavum access tool within the cannula in a controlled manner; penetrating the ligamentum flavum with the ligamentum flavum access tool to access the epidural space; and forming an opening in the ligamentum flavum with the ligamentum flavum access tool. The ligamentum flavum access tool may be any of those described above.

In one variation, a method of accessing the spine of a patient includes the steps of: anchoring the distal end of a cannula in contract with the patient's ligamentum flavum; advancing a ligamentum flavum access tool distally within the cannula in a controlled manner (wherein the ligamentum flavum access tool comprises an outer hypotube having a distal cutting edge, and an inner member comprising an atraumatic tissue contacting region that is movable within the outer hypotube, and extends distally from the outer hypotube); securing the ligamentum flavum to the atraumatic tissue contacting region of the ligamentum flavum access tool; and cutting an opening in the ligamentum flavum with the cutting edge of the proximal hypotube.

The step of securing the ligamentum flavum to the atraumatic tissue contacting region of the ligamentum flavum access tool may comprises applying a vacuum to hold the ligamentum flavum to the atraumatic tissue contacting region. In some variations, the step of securing the ligamentum flavum to the atraumatic tissue contacting region of the ligamentum flavum access tool comprises deploying one or more support elements from the atraumatic tissue contacting region when atraumatic tissue contacting region is within the epidural space. In yet other variations, the step of securing the ligamentum flavum to the atraumatic tissue contacting region of the ligamentum flavum access tool comprises penetrating the ligamentum flavum with the atraumatic tissue contacting region until the atraumatic tissue contacting region is within the epidural space as determined by the loss of resistance detector.

The step of cutting an opening in the ligamentum flavum may comprise moving the atraumatic tissue contacting region secured to the ligamentum flavum proximally so that the ligamentum flavum is cut by the cutting edge of the outer hypotube. In some variations, the step of cutting an opening in the ligamentum flavum comprises moving the cutting edge of the outer hypotube distally relative to the atraumatic tissue contacting region secured to the ligamentum flavum.

Any of these methods may also include the step of removing the ligamentum flavum access tool from the cannula.

The step of anchoring the distal end of the cannula may include removeably attaching the distal end of the cannula to the ligamentum flavum, including securing a distally anchoring cannula as described above. Alternatively (or in addition), the step of anchoring the distal end of the cannula may include anchoring the cannula to a surgical access platform.

The step of advancing the ligamentum flavum access tool may include rotating the tool relative to the cannula to advance the tool along a threaded region.

Also described herein are methods of accessing the spine of a patient comprising the steps of: anchoring the distal end of a cannula in contract with the ligamentum flavum; advancing a ligamentum flavum access tool distally within the cannula in a controlled manner (wherein the ligamentum flavum access tool comprises a proximal cutting surface, a distal tip member comprising an atraumatic tissue contacting region configured as a leading head, and a loss of resistance detector); penetrating the ligamentum flavum with the atraumatic leading head of the tip region until the atraumatic leading head accesses the epidural space as determined by the loss of resistance detector; cutting the ligamentum flavum with the proximal cutting surface; and removing the ligamentum flavum access tool from the cannula.

The step of cutting the ligamentum flavum with the proximal cutting surface may comprise compressing the ligamentum flavum between the distal tip member and the proximal cutting surface. In some variations, the step of cutting the ligamentum flavum with the proximal cutting surface comprises retracting the distal tip member so that the proximal cutting surface can engage the ligamentum flavum.

Any of the methods described herein may also include the step of deploying one or more support elements from the distal tip member when the distal tip member is within the epidural space.

Also described herein are methods of accessing the spine of a patient comprising: anchoring the distal end of a cannula in contract with the ligamentum flavum; advancing a ligamentum flavum access tool distally within the cannula in a controlled manner (wherein the ligamentum flavum access tool comprises a proximal hypotube having an expandable distal end, and a distal tip member comprising an atraumatic leading head); penetrating the ligamentum flavum with the atraumatic leading head of the tip region until the expandable distal end of the hypotube spans the ligamentum flavum; and dilating the expandable distal end of the hypotube to expand an opening in the ligamentum flavum.

The step of dilating the expandable distal end of the hypotube may include withdrawing the distal tip member proximally through the hypotube to expand the distal end of the hypotube.

The method may also include a step of removing the atraumatic leading head from the hypotube to allow access to the patient's epidural space through the cannula.

The step of penetrating the ligamentum flavum comprises determining when the distal end of the hypotube has entered the epidural space. For example, a loss of resistance detector may be used, as described.

### BRIEF DESCRIPTION OF THE DRAWINGS

FIG. 1 is a cross-sectional view of a spine, showing a top view of a lumbar vertebra, a cross-sectional view of the cauda equina, and two exiting nerve roots.

FIG. 2 is a cross-sectional view of portion of a spine and back, showing a tissue locking spinal access system in place.

FIG. 3 is a perspective view of a tissue locking spinal access system.

FIGS. 4A-4H are cross-sectional views of a portion of a spine and back, demonstrating a method for accessing a spine with a tissue locking spinal access system.

FIGS. 5A-5G are cross-sectional views of a portion of a spine and back, demonstrating a method for accessing a spine with a tissue locking spinal access system.

FIG. 6 is a posterior view of two adjacent lumbar vertebrae and an intervertebral disc, showing one example of a location for placing a spinal access cannula on vertebral bone.

FIG. 7 is a sagittal view of a portion of a lumbar spine, with a tissue locking spinal access system in place and extending through the epidural space and between adjacent intervertebral spaces.

FIGS. 8A and 8B are side views of a telescoping, tissue locking cannula system for spinal access.

FIG. 9 is a perspective view of a tissue locking spinal access system.

FIG. 10 is a perspective view of a tissue locking spinal access system.

FIG. 11 is a perspective view of an expanding, tissue locking spinal access cannula.

FIGS. 12A and 12B are side and perspective views of a curved probe/guide member system for accessing a spine through a minimally invasive cannula.

FIGS. 13A-13H illustrate the operation of one variation of a ligamentum flavum access tool, configured as a punch tool.

FIG. 13J shows different variations of ligamentum flavum access tools that are configured as punch tools.

FIG. 13K is another variation of a ligamentum flavum access tool.

FIGS. 14A-14G illustrate operation of another variation of a ligamentum flavum access tool, configured as an expander.

FIGS. 15A-15E illustrate operation of another variation of a ligamentum flavum access tool.

FIGS. 16A-16D illustrate operation of another variation of a ligamentum flavum access tool.

FIG. 16E shows different variations of ligamentum flavum access tools that are configured as barb-type tools.

FIGS. 17A-17D illustrate operation of another variation of a ligamentum flavum access tool.

FIGS. 18A and 18B show additional steps that may be performed with a ligamentum flavum access tool, such as the tool sown in FIGS. 17A-17D.

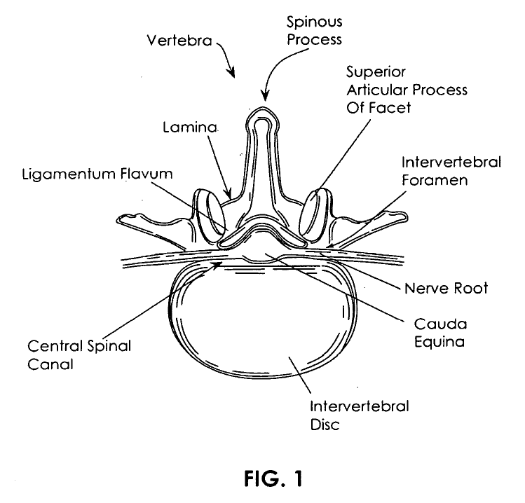

### DETAILED DESCRIPTION OF THE INVENTION

Described herein are systems, devices, tools and methods for accessing a patient's spine, and particularly a patient's epidural space. For example, described herein are tissue locking cannulas, ligamentum flavum access tools, and systems including one or both of these in addition to guide probes, guidewires, and tissue modification devices, particularly bimanual tissue modification devices.

In various embodiments, the systems, devices, and methods may be used in percutaneous, minimally invasive or less invasive surgical procedures. Alternatively, these devices, systems and methods may also be advantageous for use in an open surgical setting. While these devices, systems and methods are described primarily with reference to their uses in the spine, in some embodiments they may also be useful for accessing other parts of the body in percutaneous, minimally invasive and/or less invasive surgical procedures.

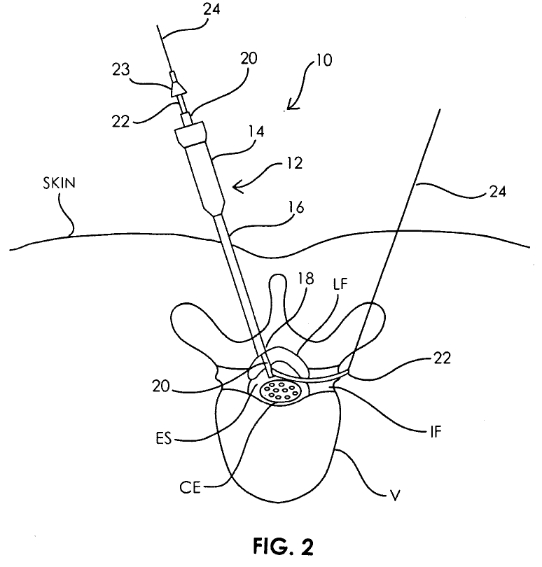

With reference now to FIG. 2, a cross-sectional view of a spine is shown, with one embodiment of a spinal access system 10 extending through a patient's skin and into the spine. Shown in the figure are a vertebra V, the cauda equina CE, the epidural space ES, two intervertebral foramina IF, and ligamentum flavum LF of the spine. In one embodiment, spinal access system 10 may include a tissue locking cannula 12, including a handle 14, a cannula shaft 16, and a tissue coupler 18 disposed at the distal end of shaft 16 for coupling with ligamentum flavum LF tissue (or additionally or alternatively periosteum of the vertebral bone and/or vertebral bone). System 10 may also include a blunt-ended probe 20, which may slide through cannula 12 and through ligamentum flavum LF to position a distal portion of probe 20 in the epidural space ES of the spine. In some embodiments, system 10 may further include a curved, cannulated, at least partially flexible guide member 22, which may slide through probe 20 to extend its curved distal portion into the epidural space ES and at least partway into (and in some cases completely through) an intervertebral foramen IF. Optionally, guide member 22 may include a pusher member 23 for facilitating advancement of guide member 22 through probe 20. In some embodiments, system may further include one or more guidewires 24, which may be advanced through a lumen of guide member 22 to extend through an intervertebral foramen IF and through the patient's skin. Alternatively, guidewire 24 may be provided separately, apart from system 10. While in some embodiments, system 10 may be used to place one or more guidewires 24 through one or more intervertebral foramina IF, in other embodiments, system 10 may be used to access an epidural space ES and possibly one or more intervertbral foramina IF for one or more other purposes, such as to provide access for a visualization device, to introduce a drug or other material or substance and/or the like.

The various components of spinal access system 10 may be made of any of a number of suitable materials and combinations of materials. For example, in some embodiments, cannula 12 may be made of a combination of stainless steel and plastic or other polymer. In some embodiments, both guide member 22 and guidewire 24 may be made of Nitinol. Alternatively, guide member 22 may be made of a polymer, such as PEEK, and guidewire 24 may be made of Nitinol. Probe 20 may be made of stainless steel, Nitinol, other metals, or any other suitable material.

In alternative embodiments, access system 10 may include fewer or additional components. For example, in one embodiment probe 20 may not be included, and guide member 22 may pass directly through tissue locking cannula 12 and partway into or through an intervertebral foramen IF. Other embodiments may include multiple guide members 22, each having a curved distal portion with a different radius of curvature to accommodate different patient anatomies. In some embodiments, pusher members 23 may be provided for any or all of probe 20, guide member 22 and guidewire 24, to facilitate passage of these components through one another. In some embodiments, these pusher members 23 may be removeably attachable, while in alternative embodiments they may be fixedly attached to their respective components.

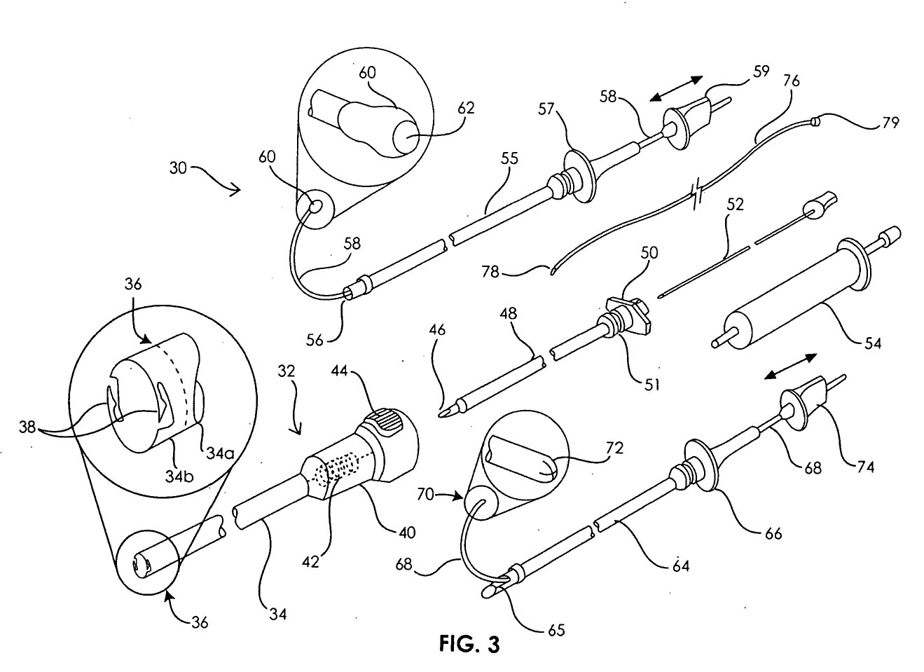

Referring now to FIG. 3, one embodiment of a spinal access system 30 is shown in greater detail. In this embodiment, access system 30 may include a number of different components, such as a tissue locking access cannula 32. Access cannula 32 may include a hollow shaft portion 34 with a tissue locking distal end 36 and a proximal hub 40 (or “handle”), which may be partially hollow and contain a spring 42 and a needle release button 44. Locking distal end 36 may, in some embodiments, include two or more tissue locking barbs 38, configured to lock into tissue when cannula 32 is rotated in one direction about its longitudinal axis and to release from tissue when cannula 32 is rotated in the opposite direction. In some embodiments, tissue locking may also require application of forwardly directed (or distally directed) pressure, to thus press and rotate cannula 32 into tissue. Alternatively, only rotation, without forwardly directed pressure, may be required in some embodiments. Barbs 38 may have any suitable shape and may range in number from two to as many as ten or more in various alternative embodiments. In fact, some embodiments may have only one barb 38 or more than ten, although multiple barbs 38 may be more efficacious than just one at attaching to tissue and more than ten barbs 38 may unnecessarily complicate manufacturing and inhibit attachment of cannula 32 to tissue. Generally, barbs 38 may all point in the same direction, relative to the circumference of cannula 32, so that turning cannula 32 in one direction attaches barbs 38 to tissue, and turning cannula 32 in an opposite direction releases barbs 38 from the tissue. In various alternative embodiments, barbs 38 may be configured to specifically attach to and release from different types of tissue. For example, in one embodiment, barbs 38 may be configured to specifically attach to ligamentum flavum tissue, in an alternative embodiment, barbs 38 may be configured to specifically attach to periosteum tissue, and in another alternative embodiment, barbs 38 may be configured to attach to both ligament and periosteum.

Other variations of the tissue locking cannula 34 may be used. For example, the tissue locking cannula may include one or more barbs or anchors that are located more proximally, either in addition, or instead, of the distal barbs illustrated and described above. For example, the tissue locking member may include anchors, hooks or barbs that are located proximal to the distal end. These anchoring members may be configured to be secured to the local spinal anatomy, and particularly the bony region (e.g., vertebra) or spinal muscle. In some variations, the anchoring members are extendable from one or more positions (e.g., ports) on the side of the cannula. For example, the anchors may be extendable from the cannula.

In one embodiment, cannula shaft portion 34 may be made of one material, such as but not limited to stainless steel. In an alternative embodiment, shaft portion 34 may be made of multiple materials joined together. For example, in one embodiment, as shown in the magnified view of tissue locking portion 36, a shaft proximal portion 34a, which may make up a majority of shaft 34, may be made of a polymer or other radio-translucent material. A smaller, distal shaft portion 34b may be made of stainless steel, some other metal, or some non-metallic radiopaque material. In use, such a multi-material shaft 34a, 34b may facilitate intraoperative radiographic monitoring of the location of shaft distal portion 34b and thus tissue locking portion 36 of cannula 32, such as by intraoperative fluoroscopy.

Spinal access system 30 may also include an epidural needle 46 coupled with a sheath 48 and proximal hub 50, which may include a lock ring 51, and a stylet 52 for residing in needle 46 as it is passed into a patient's body. Epidural needle 46 and stylet 52 may, for example, be similar to other known epidural needles and stylets presently available or hereafter conceived. Sheath 48 may cover all or a portion of needle 46 and may act to occupy space between the outer diameter of needle 46 and the inner diameter of cannula shaft portion 34, which may facilitate passage of cannula 32 and needle 46 into a patient. Needle hub 50 may fit partially within and lock into cannula hub 40, such as by means of lock ring 51, to removeably attach needle 46 and sheath 48 to cannula 32.

In one embodiment, cannula 32 may be advanced into a patient with needle 46 and sheath 48 residing within and attached to it and with stylet 52 residing within needle 46. As the epidural space of the spine is approached, stylet 52 may be removed and a syringe 54 may be coupled with needle hub 50 for performing a loss of resistance needle access of the epidural space. In one embodiment, once loss of resistance is achieved, needle 46 may be released from cannula 32 by pressing release button 44 on cannula hub 40. In some embodiments, needle 46 may be spring-loaded into hub 40, so that when release button 44 is pressed, spring 42 ejects needle 46 proximally out of cannula 32 and thus ejects the distal portion of needle 46 from the epidural space. This quick ejection method may help reduce the risk of injury to neural structures and/or dura by the sharp tip of needle 46.

In some embodiments, spinal access system 30 may also include a cannulated, at least partially rigid probe 55, which may slide through cannula 32 after needle 46 is removed. This cannulated probe may be optional. For example, a system may include just the tissue locking cannula and a flexible guide member (or simply a guide wire) may be used. Probe 55 may include a distal aperture 56, which in some embodiments may be located at the extreme distal end of probe 55, and may also include a pusher member 57 (or hub). In some embodiments, a curved, at least partially flexible guide member 58 may be provided to slide through probe 55, so that its curved distal portion extends distally out of distal aperture 56 into an epidural space of a patient. Guide member 58 may be cannulated and may include an atraumatic distal tip 60 (having a bulb shape or alternative atraumatic shapes in other embodiments) and a distal aperture 62. In some embodiments, system 30 may also include a pusher member 59 for facilitating advancement of guide member 58 through probe 55. Pusher member 57 may generally facilitate advancement of probe 55 through cannula 32. In various embodiments, pusher members 57, 59 may be either fixedly attached or removeably attachable to their respective system components. Probe pusher member 57, which may also include a hub, may facilitate attaching probe 55 to cannula 32 during use.

Probe 55 and guide member 58 may be made of any suitable material or materials. For example, in one embodiment, probe 55 may be made of a metal, such as but not limited to stainless steel, and guide member 58 may be made of a different metal, such as but not limited to Nitinol. In an alternative embodiment, guide member 58 may be made of a flexible polymer, such as PEEK. Pushers 57, 59 may similarly be made of any suitable material.

In an alternative embodiment, access system 30 may include a different probe 64 and guide member 68. In this embodiment, probe 64 may have a side-facing aperture through which curved, flexible guide member 68 passes. As mentioned above, this probe 64 (similar to the cannulated probe 55) is optional. Guide member 68 may have a blunt distal tip 70, which may not have a ball tip as in the previously described embodiment and which may have a slit opening 72 at its extreme distal end. Thus, in various embodiments, guide member 68 may have any of a number of different configurations and tip shapes. Again, probe 64 may include a pusher member 66 and/or guide member 68 may include a pusher member 74.

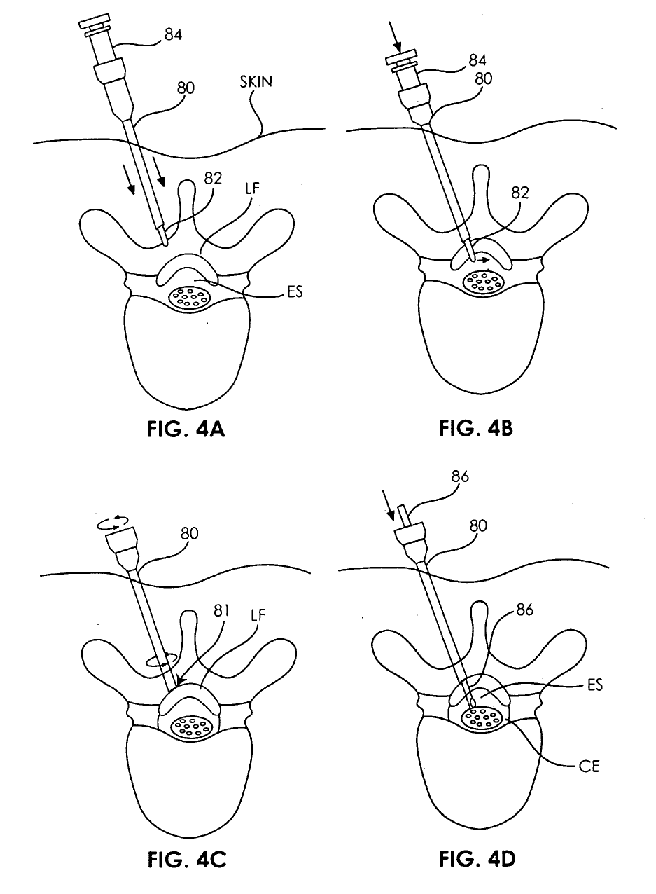

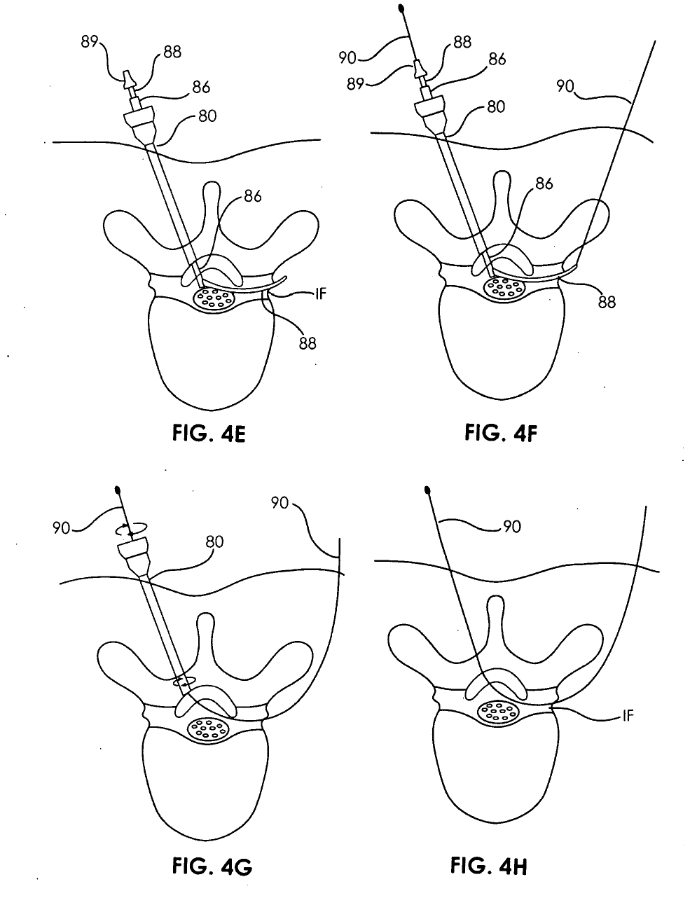

With reference now to FIGS. 4A-4H, one embodiment of a method for accessing a spine is described. As shown in FIG. 4A, in one embodiment, a tissue locking cannula 80 may be advanced through a patient's skin and into the patient over an epidural needle 82 coupled with a syringe 84. In some embodiments, cannula 80 may be advanced into the patient over needle 82 with a stylet in place through needle 82, and stylet may then be replaced with syringe 84 once needle 82 is closer to the patient's spine. Although this step is not shown in FIGS. 4A-4H, it is a known technique in epidural needle placement and may be used in some embodiments. Alternatively, in some variations, the tissue-locking cannula is advanced either by itself, or over a member such as a ligamentum flavum access tool, as described in greater detail below. Variations in which a needle is not used may be preferred, because non-needle or blunt (atraumatic) members may be less likely to damage tissue beneath the ligamentum flavum.

Nevertheless, as shown in FIG. 4B, tissue locking cannula 80 and needle 82 may be further advanced, using a loss of resistance technique, to pass a tip of needle 82 through the ligamentum flavum LF into the epidural space ES of the spine. Using a loss of resistance technique, syringe 84 will typically depress once the epidural space ES is reached with the tip of needle 82, thus passing fluid through the needle tip (solid-tipped arrows).

Once the tip of needle 82 reaches the epidural space ES, needle 82 may be quickly ejected or otherwise removed from cannula 80, thus leaving only cannula 80 in place within the patient, as shown in FIG. 4C. Cannula 80 may then be turned to lock a tissue locking distal end 81 of cannula 80 with spinal tissue, including ligamentum flavum and/or periosteum of vertebral bone. In one embodiment, needle 82 will have a length such that it will protrude a known distance out of cannula distal end 81. Thus, a surgeon or other user may know that when tip of needle 82 reaches the epidural space ES, cannula distal end 81 will reside in ligamentum flavum LF tissue. In some embodiments, placement of distal end 81 in ligamentum flavum LF and/or bone periosteum may also be confirmed by radiographic evidence, such as fluoroscopy. In alternative embodiments, cannula 80 may be locked to spinal tissue after needle 82 is removed, as shown in the figures, or alternatively may be locked to tissue before needle 82 is removed. As mentioned previously, in some embodiments, needle 82 may be removed from cannula 80 by pressing a spring-loaded release button on cannula 80 to eject needle 82 and then may be withdrawn the rest of the way out of cannula 80 manually by sliding needle 82 out.

In some variations, after securing the cannula to the ligamentum flavum, the cannula may be withdrawn slightly (proximally) so that the ligamentum flavum is “tented” by the action of the tissue locking cannula. For example, moving the tissue locking cannula proximally may help move the ligamentum flavum so that cutting or piercing the ligamentum flavum is less likely to damage underlying tissue.

In variations in which a needle or stylet is not used to penetrate the ligamentum flavum (or periostium and/or bone) before locking the tissue locking cannula, the distal end of the cannula may be placed against the target tissue, e.g., ligamentum flavum, by tactile feedback, by flurorscopic positioning, by using anatomical landmarks (such as the pedicles, etc.), or any combination of these. For example, the cannula may be advanced to the lamina by feel and/or fluoroscopy, and then walked over to the ligamentum flavum area and attached similar to FIG. 4C. Thereafter, the ligamentum flavum may be penetrated by a ligamentum flavum access device (described below) and/or a needle, stylet, or other element.

As shown in FIG. 4D, once tissue locking cannula 80 is locked to tissue, an at least partially rigid probe 86 with a blunt tip may be advanced through cannula 80, to position the probe's tip in the epidural space ES. The blunt tip of probe 86 may be configured to avoid damage to the cauda equine CE of the lumbar spine and other neural structures, such as nerve roots or spinal cord.

Referring to FIG. 4E, in one embodiment the method may next involve advancing an at least partially flexible guide member 88 through probe 86, perhaps with the help of a pusher member 89. Guide member 88 may be advanced through probe 86 to advance a curved distal portion of guide member 88 at least partway into, and sometimes all the way through, an intervertebral foramen IF of the spine.

With guide member 88 in position, and referring now to FIG. 4F, a guidewire 90 may be passed through guide member 88 and back out the patient's skin at a location apart from the entry location of cannula 80. In some embodiments, guide member 90 may have a sharp distal tip to facilitate its passage through tissue and skin and may have a shaped proximal end for coupling guidewire with a tissue modification device to be pulled into the patient. Such guidewires and methods for using them are described in greater detail, for example, in U.S. patent application Ser. No. 11/468,247, which was previously incorporated by reference.

Once guidewire 90 is placed through an intervertebral foramen IF, guide member 88, probe 86 and cannula 80 may be removed from the patient. As shown in FIG. 4G, in one embodiment, tissue locking cannula 80 may be removed by turning it in a opposite direction from the direction it was turned to lock it into the tissue. For example, if turning cannula 80 in a clockwise direction locks it to tissue, turning it in a counter-clockwise direction may unlock it from the tissue, or vice versa.

Finally, as shown in FIG. 4H, when cannula 80 and the other components of an access system are removed, guidewire 90 may be left behind to extend into the patient, through an intervertebral foramen IF and back out of the patient. Guidewire 90 may be then be used to pass an instrument into the patient's spine to perform a procedure, such as a minimally invasive decompression procedure. Again, such passage of instruments is described in greater detail, for example, in U.S. patent application Ser. No. 11/468,247. Use of various instruments to perform procedures in the spine are described in patent applications previously incorporated by reference, although the devices, systems and methods described herein are not limited to the use of such instruments.

In various alternative embodiments, the method just described may have any of a number of variations, such as fewer steps, additional steps, use of additional or different system components and/or the like. For example, in one alternative embodiment, the step of advancing probe 86 may be skipped, such that guide member 88 may be passable through cannula 80 without use of probe 86. In another alternative embodiment, probe 86 may have an articulating or bendable distal portion, and the step of advancing guide member 88 may be skipped, such that guidewire is advanced directly through probe 86, without use of guide member 88. In yet another alternative embodiment, guide member 88 may be used to deliver some other substance or structure into a spine, instead of or in addition to guidewire 90. For example, one or more pharmaceutical agents may be delivered to an intervertebral foramen IF or other area in a spine using guide member 86.

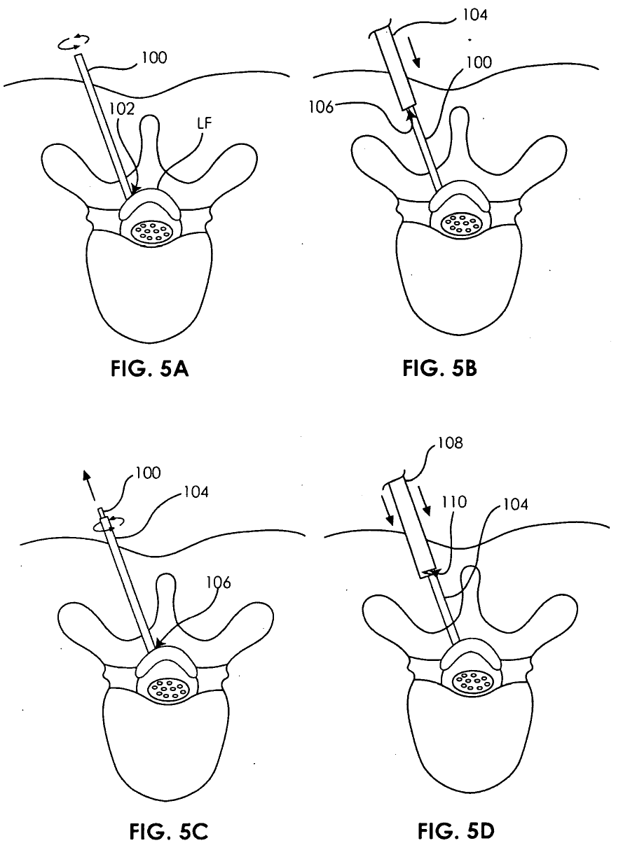

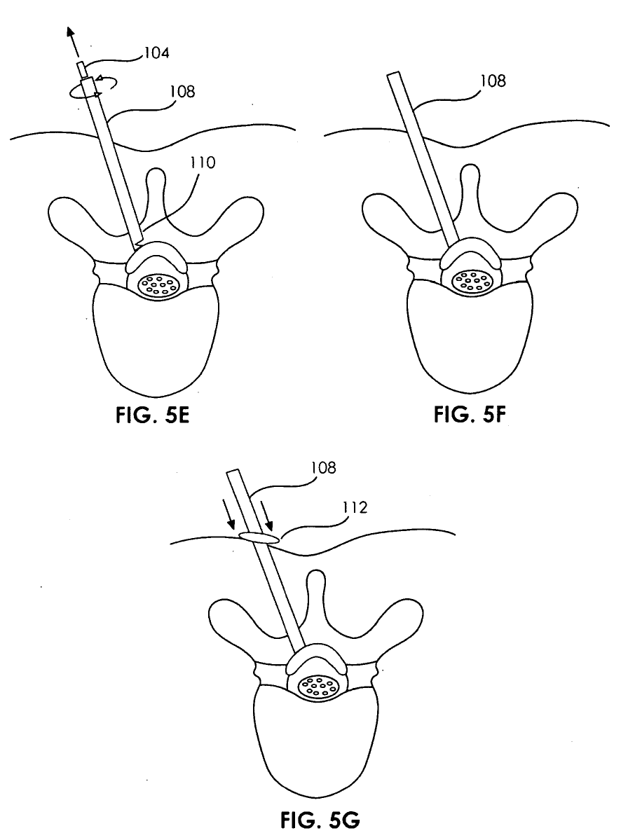

With reference now to FIGS. 5A-5G, a method for inserting an alternative embodiment of a tissue locking access cannula 100 is described. In this embodiment, cannula 100 may include a series of cannulas having different diameters, with successive cannulas being larger to slide over previously placed cannulas. Using this method, spinal access may be obtained percutaneously or with a small incision, and successively larger cannulas may then be placed to provide wider access. For example, in one embodiment, a first tissue locking cannula 100 may first be passed into the patient to contact its locking distal end 102 with ligamentum flavum LF tissue. In one embodiment, first cannula 100 may be advanced into the patient over a sylet, dilator or other device to prevent coring of tissue. Distal end 102 may lock with tissue by turning it about its longitudinal axis, as described previously. In one embodiment, a method for placing first cannula 100 may involve advancing it into a patient until distal end 102 contacts a vertebral lamina and then moving (or “walking”) distal end 102 gradually off the lamina until it reaches soft tissue—i.e., ligamentum flavum LF. In an alternative embodiment, distal end 102 may be attached to periosteum of a vertebral lamina. In yet another alternative embodiment, distal end 102 may be attached to both ligamentum flavum and periosteum.

As shown in FIG. 5B, once first cannula 100 is attached to ligamentum flavum LF (and/or other tissue), a second cannula 104, also having a tissue locking distal end 106, may be passed over it into the patient. In FIG. 5C, second cannula 104 is turned to lock its distal end 106 to ligamentum flavum LF, and first cannula 100 may then be removed. In alternative embodiments, first cannula 100 may be removed before attaching second cannula 104 to tissue.

In FIG. 5D, a third tissue locking cannula 108 with a tissue locking distal end 110 is advanced over second cannula 104. In FIG. 5E, third cannula is turned to attach it to ligamentum flavum LF and second cannula 104 is removed through third cannula 108, leaving third cannula 108 attached to tissue and extending out of the patient, as shown in FIG. 5F. In some embodiments, as in FIG. 5G, the last cannula inserted, such as third cannula 108, may include a sliding tissue lock 112 for coupling cannula 108 to a patient's skin. Such a tissue lock 112 may be part of a cannula or, in alternative embodiments, it may be a separate component slideable over a cannula. Such a sliding tissue lock 112 may help stabilize a cannula 108, since it would be attached to tissue inside the patient and locked at the skin as well.

In various embodiments, the method described in FIGS. 5A-5G may include any number of sliding cannulas, such as but not limited to between two and ten cannulas. Each cannula may be made of any suitable material or combination of materials, such as but not limited to stainless steel, Nitinol, other metal, polymer, ceramic or the like. In some embodiments, cannulas 100, 104, 108 may be used with a spinal access system such as the one described in FIG. 3. Once access is achieved, any suitable procedure may be performed, such as but not limited to a minimally invasive spinal decompression procedure like those described in patent applications incorporated previously by reference.

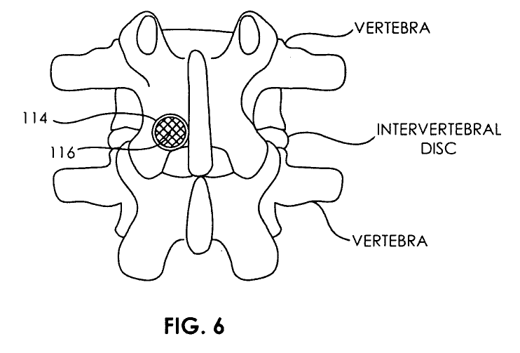

Referring now to FIG. 6, in an alternative embodiment, a tissue locking cannula device 114 may be inserted into a patient and attached to periosteum of a vertebral bone, such as periosteum covering a vertebral lamina. In such an embodiment, cannula device 114 may be used to provide an access window 116 through which a laminotomy may be performed, thus providing access through a lamina to the epidural space. FIG. 6 illustrates a posterior view of two adjacent vertebrae with one example location for placement of cannula device 114 and window 116. In alternative embodiments, any number of other locations may be used for placement of device 114. In one embodiment, a portion of device 114 may be placed oveverertebral bone, attaching to periosteum, and portion may be placed over an intervertebral space, attaching to ligamentum flavum.

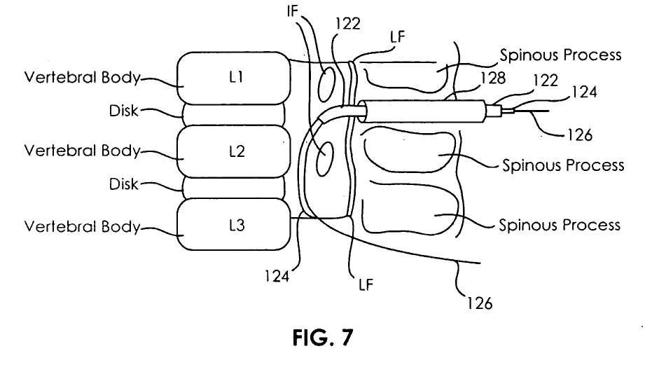

With reference now to FIG. 7, in an alternative embodiment, a tissue locking cannula 120 and other access system components may be used to access a spine and pass a guidewire through a space between two vertebrae, through the epidural space, and back out of the spine through a space between two different vertebrae (or one of the first vertebrae and an adjacent vertebra). For example, in FIG. 7, locking cannula 120 is passed between the L1 and L2 vertebrae and attached to ligamentum flavum LF tissue. A probe 122 extends through cannula 120, a guide member 124 extends through probe 122, and a guidewire 126 extends through guide member 124. Rather than extending into or through an intervertebral foramen IF, guide member 124 passes into the epidural space, through ligamentum flavum LF and between the L2 and L3 vertebrae. Guidewire 126 thus passes into the spine between L1 and L2 and out of the spine between L2 and L3. In one embodiment, guidewire 126 may then be used to advance a tissue removal device into the spine to remove tissue to treat central spinal stenosis.

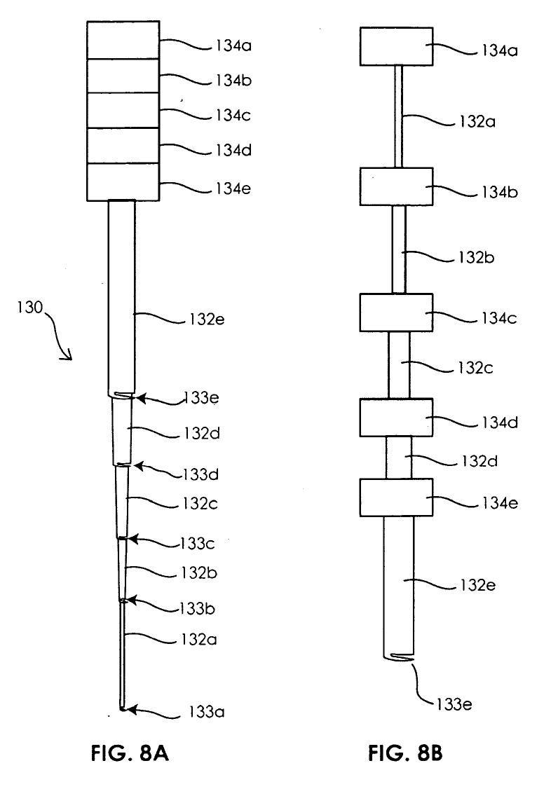

Referring now to FIGS. 8A and 8B, in one embodiment, multiple tissue locking cannula may be slideably coupled in a telescoping, tissue locking cannula system 130. In one embodiment, cannula system may include multiple cannulae 132, each having at least two tissue locking barbs 133 at its distal end, and each having a small handle or stop 134 at its proximal end. In various embodiments, any number of cannulae 132 may be included, such as but not limited to between two and ten cannulae, or as shown in FIGS. 8A and 8B, five cannulae 132. Each cannula 132 may have any desired diameter, ranging for example from between about 1 mm and about 30 mm in diameter, or more preferably ranging from between about 1 mm and about 20 mm.

In any of the variations described herein, the barbs or anchors may be configured so that they do not completely penetrate the tissue. For example the barbs (or other anchoring members) may be configured so that they removeably attach. For example, the barbs may only shallowly attach to the ligamentum flavum, protecting the tissue (e.g., nerves, etc.) below the ligamentum flavum from potential damage by the anchoring members. For example, the barbs may be configured to penetrate less than 2 mm, less than 1.5 mm, less than 1 mm, etc. into the ligamentum flavum. In some variations, the barbs are configured so that they are limited from extending deeply. For example, the barbs may be shaped or angled so that they only shallowly penetrate the tissue such as the ligamentum flavum.

In use, cannula system 130 may be used in a spinal access method similar to the one described in FIGS. 5A-5G. System 130 may be advanced into a patient in a configuration such as in FIG. 8A, with a first, smallest diameter cannula 132a in a front (or most distal) position. First cannula 132a may be locked into (attached to) tissue such as ligamentum flavum by rotating a first handle 134a, and then a second cannula 132b may be advanced over it and locked into the tissue by rotating a second handle 134b. Once second cannula 132b is locked to tissue, first cannula 132a may be released from the tissue by rotating it in an opposite direction from the locking direction and then withdrawing first cannula 132a from system 130. Alternatively, first cannula 132a may be left in place and removed later in the access process. Third 132c, fourth 132d and fifth 132e cannulae may be advanced in succession in the same manner, with each smaller cannula being removed when the next largest cannula is attached to tissue. Thus, a path into the patient's tissue and to the spine is gradually dilated, until a largest diameter cannula 132e is in place and attached to ligamentum flavum, periosteum and/or other spinal tissue. Any of a number of procedures may then be performed through cannula 132e, such as but not limited to a spinal decompression procedure and/or a spinal fusion.

FIG. 8B shows a configuration of telescoping, tissue locking cannula 130 in which the smaller cannulae 132a-132d have been partially withdrawn from the largest diameter cannula 132e. As mentioned above, in various embodiments, each cannula 132 may be removed individually when the next largest cannula has been placed, or alternatively all cannulae 132 may be placed before removing the smaller cannulae.

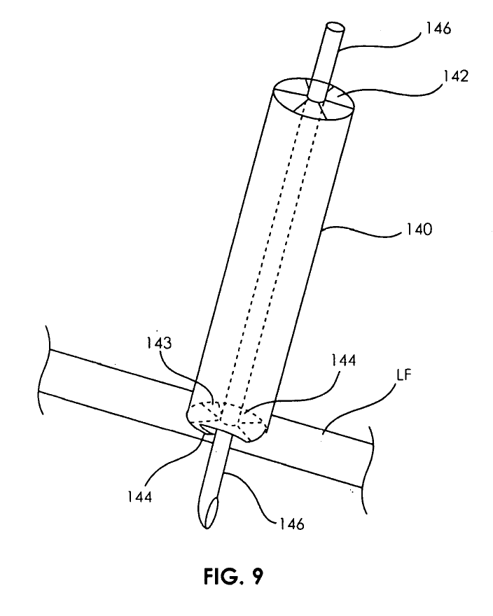

Referring now to FIG. 9, in another embodiment, a tissue locking cannula 140 may include multiple tissue locking barbs 144, as have been described previously, and at least one of a proximal port 142 or a distal port 143 for helping guide an epidural probe 146 (or epidural needle) through cannula 140 an into an epidural space. As shown, cannula 140 may be attached to tissue such as ligamentum flavum LF, and probe 146 may be advanced through cannula 140, with ports 142, 143 helping to guide probe 146 in a desired orientation. In alternative embodiments, only proximal port 142 or only distal port 143 may be included. Ports 142, 143 may be made of a flexible material, such as a polymer, to create a structure similar to a flap, or alternatively they may be made of a rigid material such as a metal.

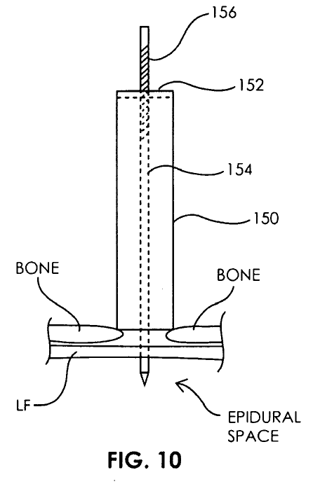

With reference to FIG. 10, in another embodiment, a tissue locking cannula 150 may be coupled with bone periosteum and may include a port 152 for guiding a needle 154 or probe. In one embodiment, needle 154 (or probe) may include threads 156 which fit with complementary threads on port 152, thus allowing needle 154 to be threaded/screwed into cannula 150. Such threads 156 may facilitate gradual, controlled advancement of needle 154 into the epidural space. In some embodiments, as illustrated in FIG. 10, cannula 150 may be attached to periosteum of adjacent vertebral bones, such as the laminae of adjacent vertebrae, and needle 154 or probe may be advanced through ligamentum flavum LF into the epidural space. In alternative embodiments, cannula 150 may be attached to periosteum of one lamina, to ligamentum flavum LF, or to periosteum and ligamentum flavum LF.

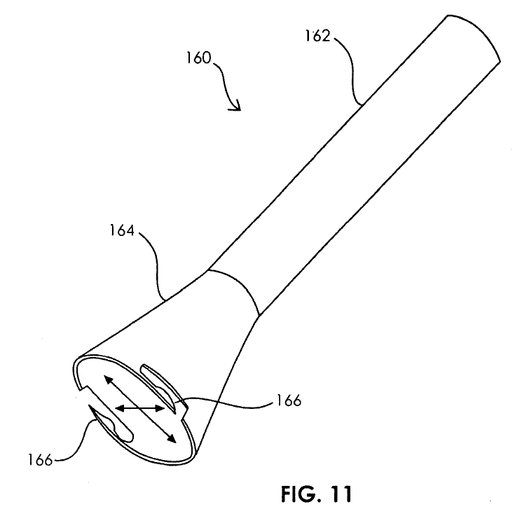

Referring now to FIG. 11, in another embodiment, a tissue locking spinal access cannula 160 may include a proximal tubular portion 162 and a distal expandable portion 164 including multiple tissue locking barbs 166. Products such as the Atavi® Atraumaic Spine Surgery System (provided by Zimmer Holdings, Inc., Warsaw, Ind.) provide a cannula with an expanding distal portion but do not provide for locking with internal patient tissue. Cannula 160 combines the convenience of expandable distal portion 164 with tissue locking barbs 166 to help stabilize the device 160 within the patient.

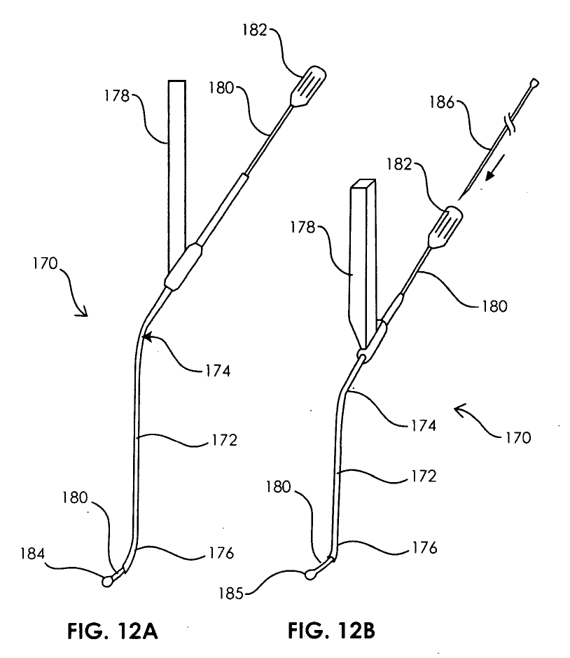

With reference now to FIGS. 12A and 12B, in one embodiment, a spinal access probe system 170 may be configured for use through a minimally invasive access cannula, such as one or more of the cannulae described above and/or currently available cannulae, such as but not limited to the Atavi® Atraumaic Spine Surgery System (referenced above) or the Medtronic METRx™ MicroDiscectomy System (Medtronic, Inc., www.medtronic.com). In many cases, instruments to be used through such a minimally invasive cannula system may benefit from being curved or bayoneted, so a surgeon's view through the cannula will not be blocked by the instruments. Thus, in one embodiment, probe system 170 may include a curved (or “bayoneted”) probe 172, including a proximal bend 174 and a distal bend 176, as well as a handle 178. A curved guide member 180, such as those described previously above, may slide through probe 172 and may include a proximal handle 182 and an atraumatic distal tip 184. As shown in FIG. 12B, atruamatic tip 184 may include an aperture 185. A guidewire 186 may be passed through guide member 180 to pass out of aperture 185, as has been described previously. Also as described previously, the various components of system 170 may be made of any suitable material or combination of materials, such as but not limited to stainless steel, Nitinol, other metals, polymers and the like.

In addition to those described above, other spinal access devices, systems and methods are also described and illustrated below, and any of these devices and systems may be used with any of those described above. For example, any of the ligamentum flavum access tool devices described herein may be used with one or more of the removeably attachable tissue locking cannula.

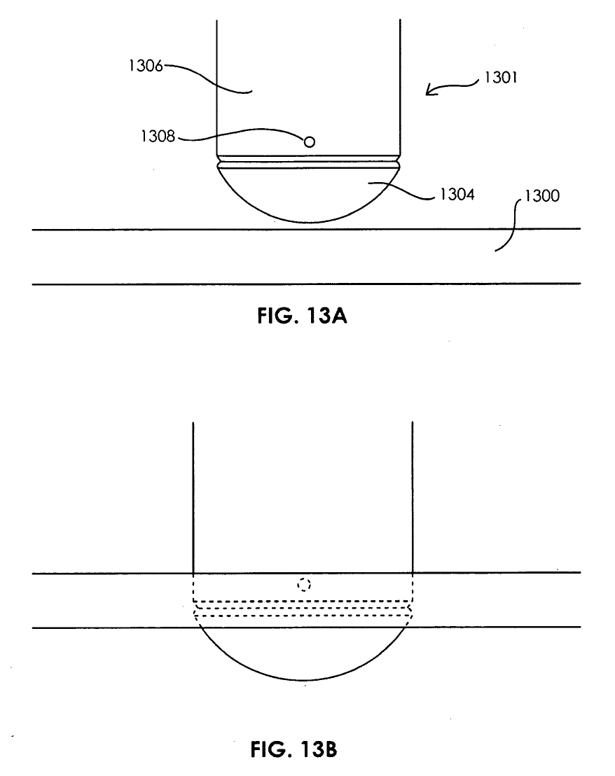

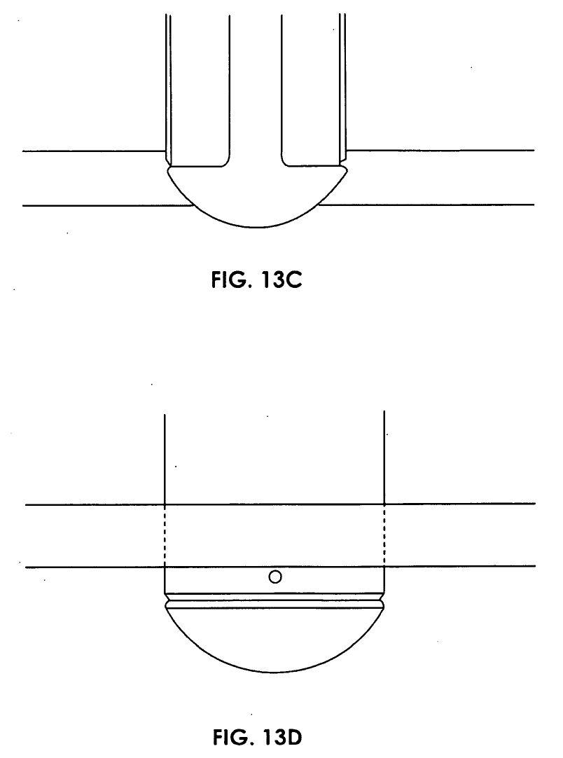

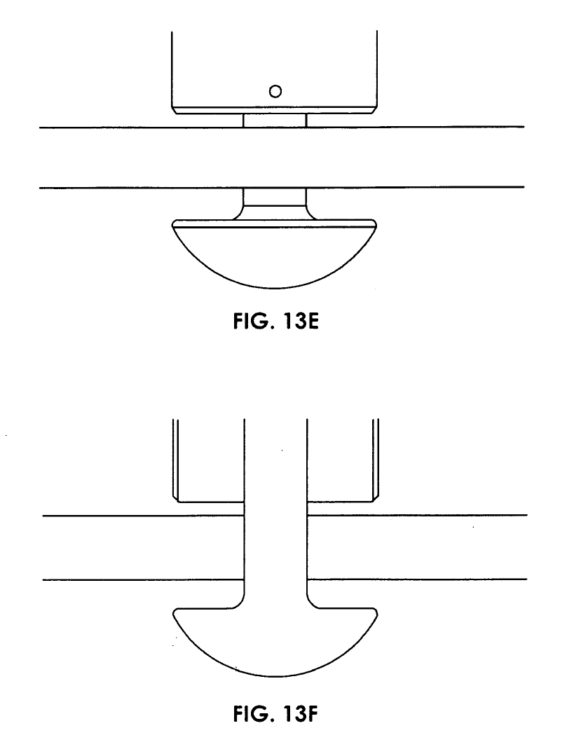

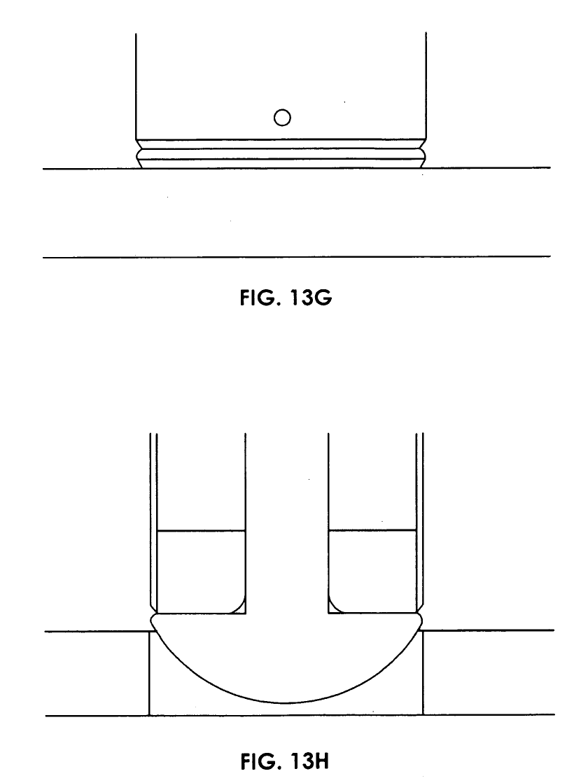

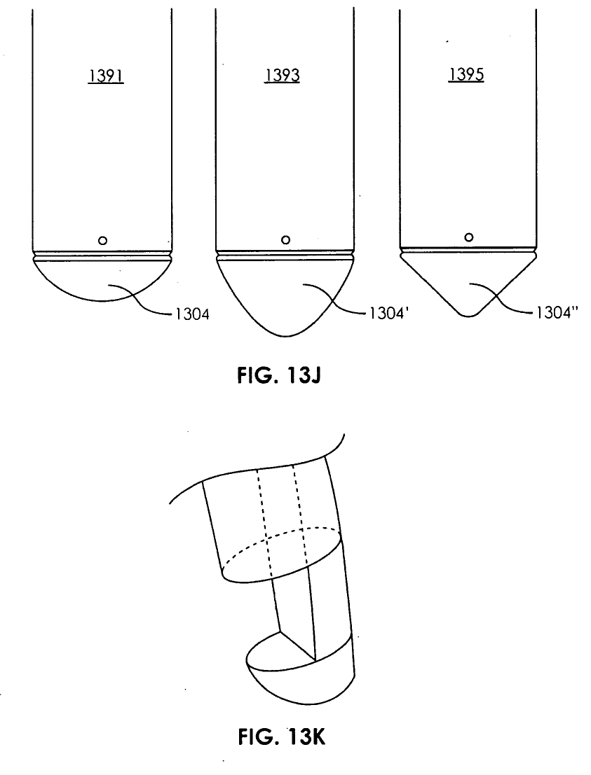

For example, FIGS. 13A to 17D illustrate five variations of ligamentum flavum access tools and methods of using them to access a patient's epidural space. Any of the features or elements of these exemplary variations may be used with any of the other exemplary variations.

In general, a ligamentum flavum access tool may atraumatically access a patient's epidural space. These devices may include an outer hypotube (i.e., cannula) member and an inner member that is axially movable relative to the outer member. The inner (atraumatic) member typically extends distal to the outer member. In some variations the outer member is sharpened. For example, the outer member may be a cannula having a sharp or cutting edge. The inner member, the outer member or the combination of the two may have an atraumatic tip (e.g., domed, blunt, mushroom-shaped, etc.). The device (and particularly the outer member) may be advanced in a controlled fashion, and is configured so that the user does not axially advance the device towards the dura. For example, the device may be anchored (e.g., directly to the patient or to a surgical access platform) and advanced by a rotary (e.g., screwing) motion. For example, the device or a portion thereof may be threaded on an outer surface so that rotating the device in a first direction causes it to advance. In some variations, the device may be geared so that the rate of advancing and/or retraction of the device towards the dura may be even more finely controlled.

The device may also include one or more detectors for detecting when the device (or a portion of the device) has penetrated the ligamentum flavum. For example, the device may include a hole or opening in the device for detecting a loss of resistance once the device has penetrated the ligamentum flavum.

FIGS. 13A-13H illustrate one variation of a ligamentum flavum access tool, configured as a ligamentum flavum punch. FIG. 13A illustrate a perspective view of the ligamentum flavum access tool 1301 approaching the ligamentum flavum 1300. For the sake of simplicity, this example shows the tool approaching without any additional guide. In use, the tool may be applied within a cannula or other guide. For example, the ligamentum flavum access tool may be applied within a tissue locking cannula that has been secured to the ligamentum flavum, as illustrated and described above (e.g., instead of a penetrating needle, the system may include a ligamentum flavum access tool used in any of the ways described above). Thus, a cannula may be placed attached to (or adjacent) the ligamentum flavum and used to deliver the ligamentum flavum access tool. Alternatively, the ligamentum flavum access tool may approach the ligamentum flavum without the benefit of an additional guide.

The ligamentum flavum access tool in FIG. 13A includes a distal blunt head region 1304 and a more proximal outer cannula 1306. A loss of resistance detector is located on the proximal outer cannula 1306.

In this variation, the ligamentum flavum access tool includes an atraumatic leading tip 1304 that is similar to a mushroom head to minimize trauma to dura during penetration of ligamentum flavum (LF). An alternate tip design could match the profile of a Penfield 4, a dissector with a thin-profile, atraumatic tip that is commonly used to penetrate the LF. Immediately proximal to the leading tip in this example, is a hypotube (proximal cannula) 1306 with a sharpened edge and a distal side hole 1308 for loss of resistance detection once the device has penetrated the LF.

The entire assembly may be advanced through the LF and into the epidural space in a controlled fashion. For example, the ligamentum flavum access device may be advanced using a screw thread system. In this variation, the device may be anchored to the patient (or to a surgical access platform). The user does not apply axial force (towards the dura) to gain access to the epidural space. Instead, the distal tip is advanced with a screw thread which provides a controlled and consistent movement of the tip through the LF. As the device is advanced, the atraumatic tip and sharpened hypotube move together as a single unit through the LF and into the epidural space. Epidural access is detected through the side port in the hypotube using the loss of resistance technique. This is illustrated in greater detail in FIGS. 13B and 13C (showing a cross-section through the device).

In this example, once epidural access has been achieved, as shown in FIG. 13D, the leading tip (e.g., a mushroom head or Penfield 4 profile or any other appropriate profile) is held fixed within the epidural space while the sharpened hypotube is retracted proximally and withdrawn to the exterior of the LF, as shown in FIG. 13E. In some variations, only the distal inner member is advanced while the proximal hypotube remains outside of the ligamentum flavum. In such variations, it may be useful to have the loss of resistance input (opening 1308) on the distal head region.

Once the sharpened hypotube is completely outside of the LF, as shown in FIGS. 13E and 13F, the hypotube is rigidly fixed in this position. As a result, the LF is sandwiched between the leading tip (proximal surface) and the sharpened hypotube.

As shown in FIGS. 13E and 13H, the leading tip may be pulled back proximally towards the hypotube thereby tenting the LF and pushing it against the sharpened hypotube edge. In this way, a hole in the LF is created through this punching action. In addition, the plug of removed LF will be captured within the hypotube and not be lost within the epidural space.

Other variations of ligamentum flavum access tools are shown side-by-side in FIG. 13J. The variation shown on the far left 1391 is the ligamentum flavum punch tool shown in FIGS. 13A- 13H, in which the distal end of the tool (the distal blunt head region) is configured as a blunt, essentially mushroom-shaped cross-section. The variation 1393 shown in the middle of FIG. 13J has a slightly more tapered head 1304′, which is bullet shaped. This variation is otherwise similar to the variation illustrated in FIGS. 13A-13H, and may otherwise be used the same. Similarly, the variation shown in the far right 1395 of FIG. 13J is even more steeply tapered, and has a conical or silo tip 1304″ on the distal blunt head region. Any of the distal head regions described herein may be shaped as shown in FIG. 13J, or other similar blunt shapes.

Any of the ligamentum flavum access tools described herein may also be configured so that they have an asymmetric cutting shape. FIG. 13K illustrates one variation of a ligamentum flavum access device that includes an asymmetric cutting region. This example is similar to the variations shown above (e.g., in FIGS. 13A-13J), and includes a distal head region (blunt head region) that is configured to extend into the ligamentum flavum. The head region in FIG. 13K also attaches to an elongate member (e.g., cannula, wire, rod, etc.), but attaches asymmetrically, so that, rather than a “mushroom shape” as shown, the distal head has an opening more to one side of the device than the other. This allows the ligamentum flavum to extend beneath the head of the device as described above, e.g., in FIG. 13F, however it extends asymmetrically. In this variation, and in similar variations, the cut in the ligamentum flavum may not be round, but may have a profile that is oval, half-circle, crescent, or other shapes.

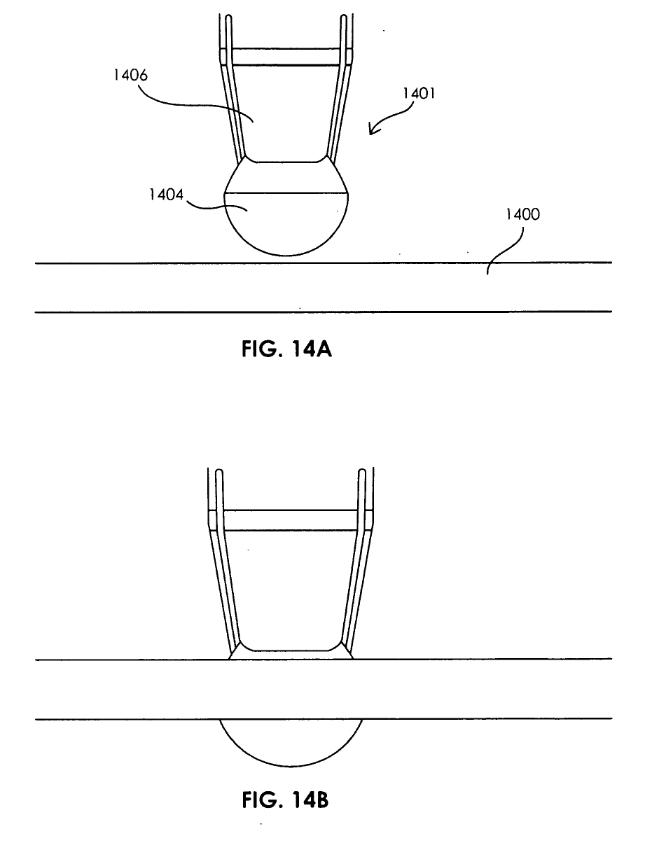

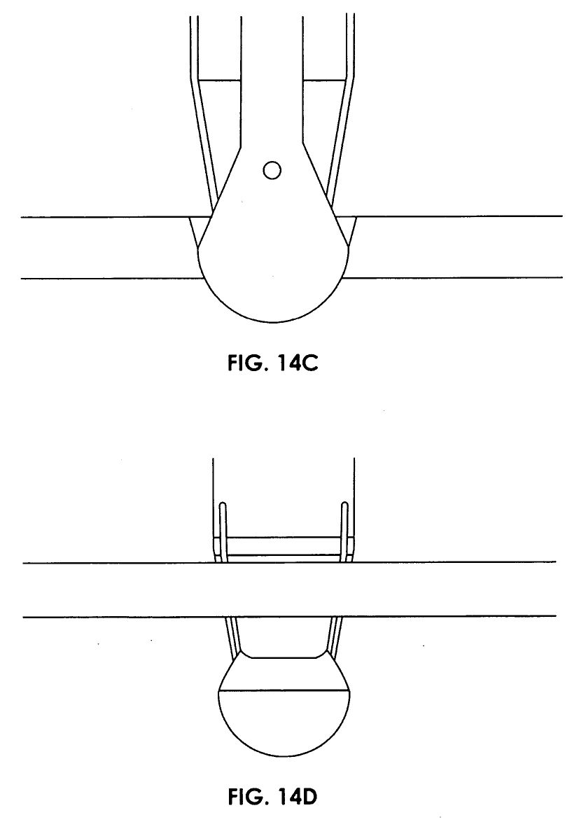

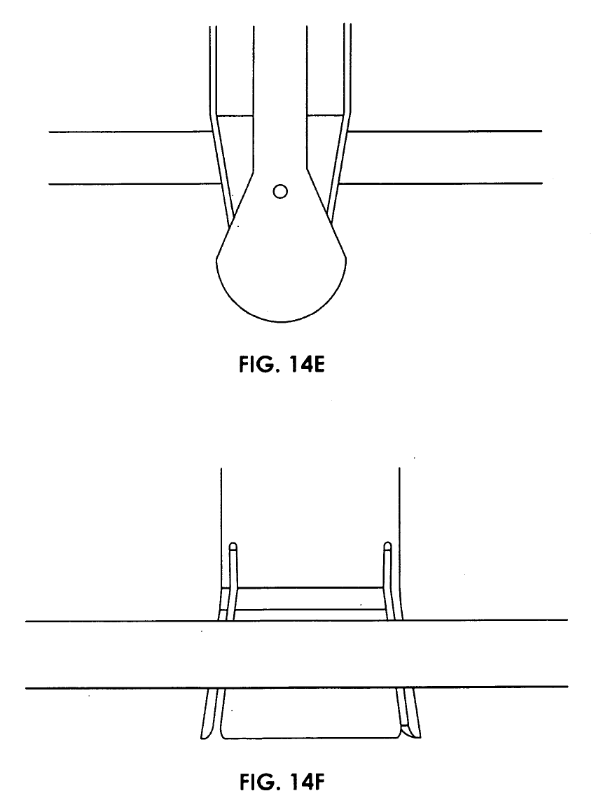

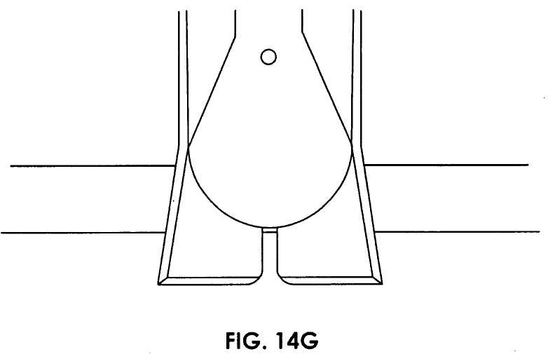

FIGS. 14A-14G illustrate the operation of another variation of a ligamentum flavum access device. In this variation the access device is configured as a ligamentum flavum dilator.

FIG. 14A shows this variation of a ligamentum flavum access device 1401 prior to penetrating the ligamentum flavum 1400. The distal-most member of this variation is configured as an atraumatic leading tip 1404 similar to mushroom head to minimize trauma to dura during penetration of ligamentum flavum (LF). An alternate tip design would closely match the profile of a blunt spherical shape. Proximal to the end of the device, there is a distal side hole for loss of resistance detection once the device has penetrated the LF. Immediately proximal to the leading tip is a hypotube 1406 with axial slits at the distal tip.

As illustrated in FIG. 14B and 14C, the entire assembly is advanced through the LF and into the epidural space, in a controlled fashion, e.g., using a screw thread system. The entire device may be anchored to the patient (or to a surgical access platform). The user does not apply axial force (towards the dura) to gain access to the epidural space. Instead, the distal tip is advanced with a screw thread which provides a controlled and consistent movement of the tip through the LF. As the device is advanced, the atraumatic tip and slit hypotube may move together as a single unit through the LF and into the epidural space. Epidural access is detected through the side port in the leading tip using the loss of resistance technique.

Any of the devices described herein may include a sensor to determine epidural access. In addition to the loss of resistance technique sensors mentioned, other sensors (pressure, resistance, force, biomarker, etc.) may be used. In some variations the sensor may be electronic.

As shown in FIG. 14D, the entire device (assembly) may be inserted until the expandable distal region of the outer member is within the ligamentum flavum. Once epidural access has been achieved, as shown in FIG. 14E, the leading tip (mushroom head or blunt, spherical shape) may be retracted proximally while the slit hypotube is held fixed within the epidural space.

As shown in FIG. 14F and (in cross section) 14G, the process of pulling the blunted tip through the center of the slit hypotube causes the distal end of the tube to flare open under the wedging action of the tube ID to tip interface. The flaring open of the distal end of the hypotube within the LF dilates the entry site and expands the opening in the LF. This concept takes advantage of a small, atraumatic entry through the LF and subsequent dilation of this entry point to provide adequate access to the epidural space, as shown.

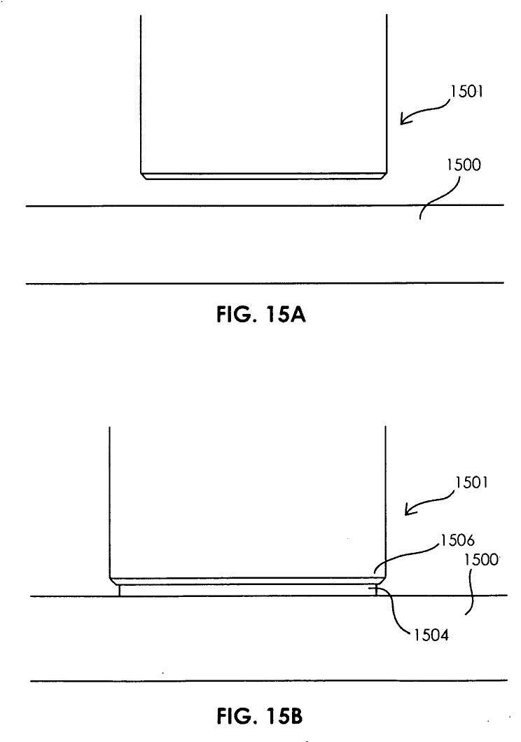

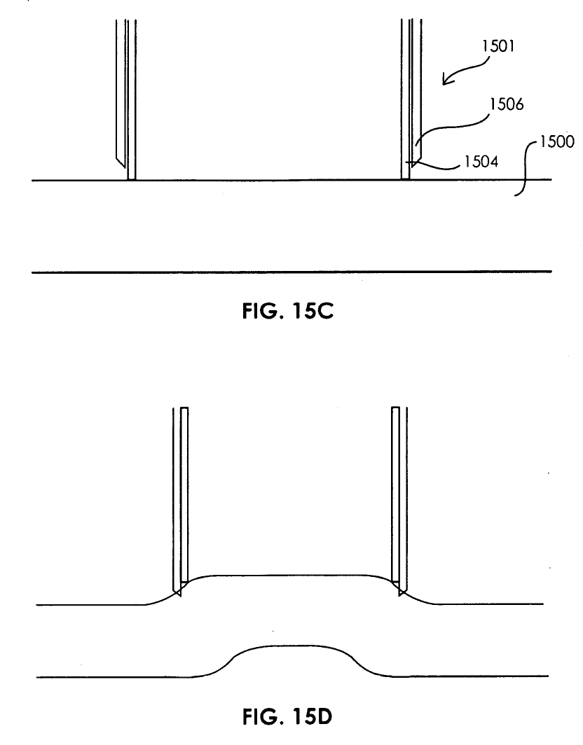

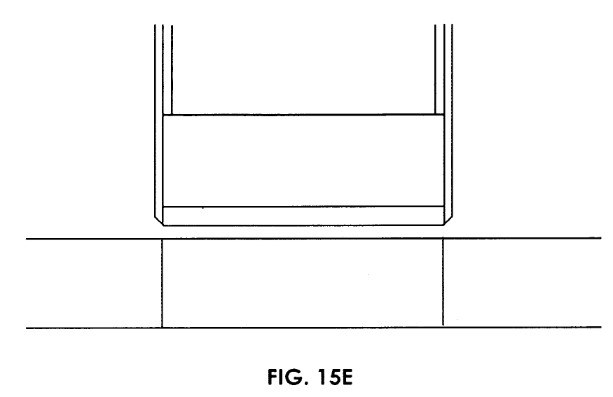

FIGS. 15A-15E illustrate another variation of a ligamentum flavum access device, configured as a vacuum device.

For example, FIG. 15A shows a side view of this variation. The distal end of device 1501 is comprised of 2 close-fitting, concentric hypotubes. The outer hypotube 1506 has a sharpened edge. The entire assembly may be advanced to the LF 1500 and be docked to the LF outer surface. The entire device can be anchored to the patient (or to a surgical access platform). Thus, as with the other variations described, the user does not apply axial force (towards the dura) to advance the device against the LF. Instead, the distal tip is advanced with a screw thread which provides a controlled and consistent movement of the tip to the LF.

As shown in FIGS. 15B and (in cross section) in FIG. 15C, the device is advanced, and in this example the concentric hypotubes 1504, 1506 move together as a single unit. Once contact with the LF is achieved, the axial position of the sharpened outer tube is held fixed.

Vacuum may then be drawn through the inner tube thereby drawing the outer surface of the LF to the inner tube, as shown in FIG. 15D. With the outer tube held fixed axially, the inner tube is drawn biased proximally under the influence of a spring. In this way, the inner tube is pulling the LF against the outer tube sharpened edge. With the LF pulled against the outer tube, the outer tube can be rotated in place. This rotation slices into the LF. As the LF is cut, the spring tension on the inner tube draws the LF further into the outer tube to advance the cutting depth into and eventually through the entire LF. Completion of cutting through the LF can be detected when the inner tube no longer encounters resistance against the proximally directed spring bias. At this point, a hole has been cut through the LF and epidural access has been gained. This is illustrated in FIG. 15E.

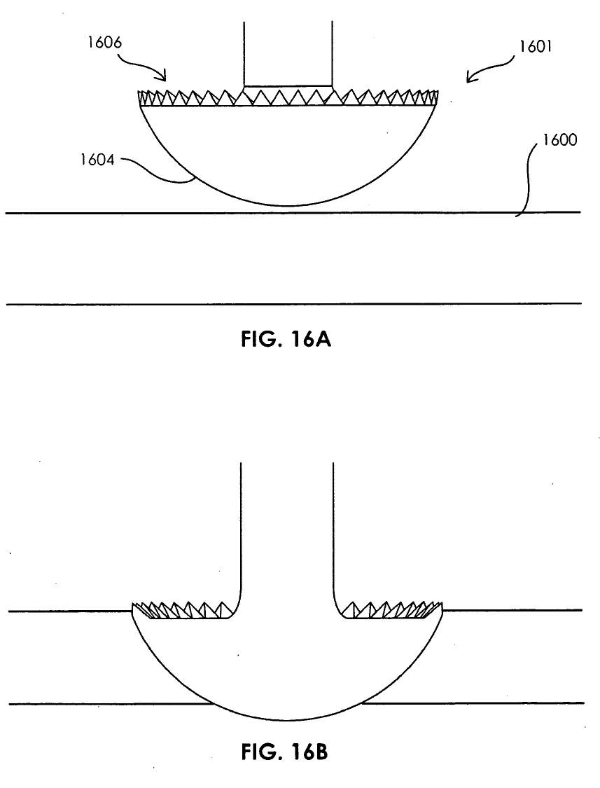

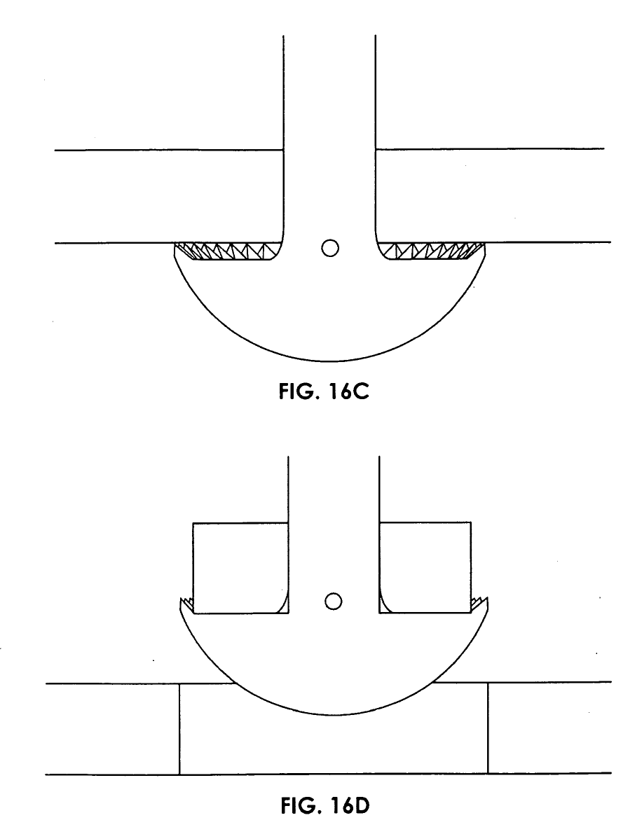

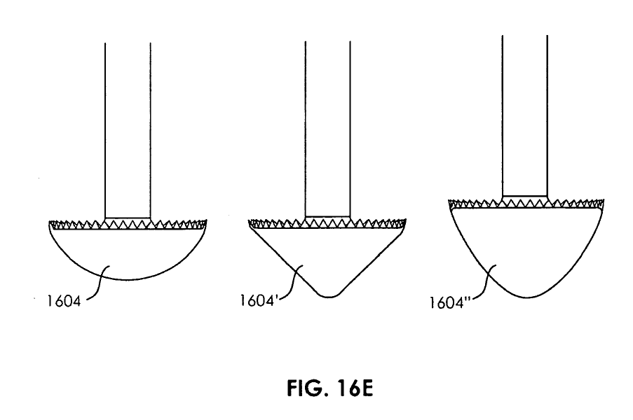

FIGS. 16A-16D illustrate another variation of a ligamentum flavum access device, configured as a ligamentum flavum barb. In FIG. 16A the ligamentum flavum access device 1601 includes an atraumatic leading tip 1604 (which may be shaped similar to a mushroom head) to minimize trauma to dura during penetration of ligamentum flavum (LF) 1600. An alternate tip design could match the profile of a Penfield 4, as described above. In this variation, the proximal surface 1606 of the leading tip is a cutting edge, and may be barbed, serrated, or the like to enable tearing or cutting of the of the ligamentum flavum.

A hypotube extends immediately proximal from the leading tip, and may include a distal side hole for loss of resistance detection once the device has penetrated the ligamentum flavum. Alternatively, one or more other sensors for determining when the device has penetrated into the epidural space may be used.

The entire assembly may be advanced through the ligamentum flavum, and into the epidural space in a controlled fashion, as illustrated in FIG. 16B. The device may be controllably advanced, as previously described. For example, the device may be advanced using a screw thread system. The entire device may be anchored to the patient (or to a surgical access platform), so that the user does not apply axial force towards the dura to gain access to the epidural space. Instead, the distal tip may be advanced with a screw thread which provides a controlled and consistent movement of the tip through the ligamentum flavum. As the ligamentum flavum access device is advanced through the ligamentum flavum, the ligamentum flavum may be tented and stretched as the device passes through.

As the device is advanced, the atraumatic tip may be moved through the ligamentum flavum 1600 and into the epidural space, as shown in FIG. 16C. The head of the device may penetration into the epidural space so that the proximal cutting surface is within the epidural space. Advancing the device may stop once the epidural space entry is detected. For example, epidural access may be detected through the side port in the hypotube behind the leading tip using the loss of resistance technique. Once epidural access has been achieved, the leading tip (mushroom head or Penfield 4 profile) can be retracted proximally, engaging the barbs of the proximal cutting surface against the inner surface of the ligamentum flavum.

After the ligamentum flavum is engaged by the barbs, the device may be pulled in the proximal direction tearing a hole in the ligamentum flavum as it is withdrawn, as shown in FIG. 16D. In this way, an opening for epidural access has been created. The device may be rotated or moved to assist in cutting the ligamentum flavum.

FIG. 16E shows other variations of the ligamentum flavum barb-type access devices, including the variation illustrated in FIGS. 16A-16D. The variation shown on the far left of FIG. 16E, 1691, is identical to the variation illustrated in FIGS. 16A-16D. The variation of a ligamentum flavum barb shown in the middle of FIG. 16E, 1693, has a conical or silo tip 1604′. Similarly, the variation shown in the far left of FIG. 16E has a bullet shaped tip 1604″. Any of these devices may be used as illustrated and described above for FIGS. 16A-16D.

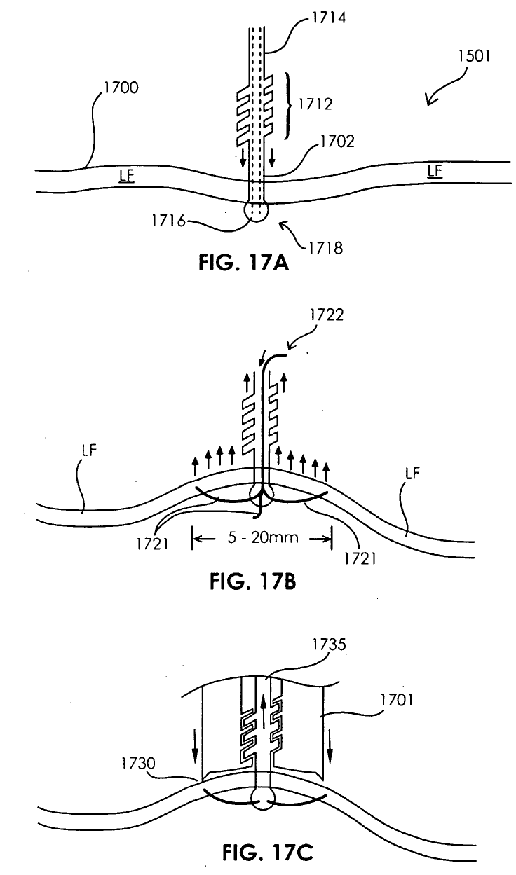

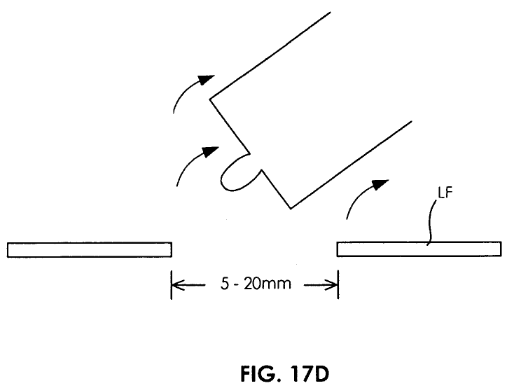

FIGS. 17A-17D illustrate another variation of a ligamentum flavum access device, configured to expand within the epidural space, and support the ligamentum flavum so that it can be cut. In this variation an inner member 1704 includes a distal atraumatic portion 1706 that is configured as a rounded tip region 1718 that has at least one detector for determining when the distal tip has accessed the epidural space. In the example shown in FIG. 17A, the distal tip 1718 has an opening 1716 for loss of resistance detection. An elongate neck region extends from the distal tip 1718 of the epidural access device 1702, and includes a threaded region 1712 which may mate with a cutting element (e.g., a cannula including a cutting edge), as illustrated in FIG. 17C. The elongate neck region may then continue proximally 1714.

One or more extendable support elements are extendable from the distal portion 1718 of the inner member 1706 when the inner member is within the epidural space. For example, the inner member may include one or more arms that extend from the distal region of the inner member after it has passed into the epidural space. In some variations the support element(s) are arms made of Nitinol or other shape-memory or appropriately deformable material that may be extended from the inner member (e.g., substantially perpendicular to the long axis of the inner member). FIG. 17B illustrates the extension of three support members.

In FIG. 17B, three support members 1721 are deployed from the distal end of the inner member 1702 by pushing an expandable inner member from the lumen of the inner member. For example, a wire, pushrod, or other element 1722 may be used to deploy the extendable members from the distal end of the inner member. The deployable member(s) may also be retracted into the inner member by pulling up (e.g., on element 1722). As illustrated in FIG. 17B, the support elements 1721 may be expanded from the inner member in a direction that is substantially perpendicular from the inner member. For example, the exit openings on distal region of the inner member may be oriented on the sides of the device. In some variations the support member is pre-biased so that extends approximately perpendicularly from the inner member.

Thus, the support member(s) may be configured to extend into the epidural space without damaging nearby structures, and may extend under the ligamentum flavum so that it can be supported during cutting. In some variations the support members are atraumatic support members, and may include non-sharp (e.g., rounded, etc.) distal ends or other surfaces.

After deploying the support member(s) from the inner member 1702, an outer member 1701 may be applied to cut the ligamentum flavum, as illustrated in FIG. 17D. In this example, the outer member 1701 is configured as a hypotube (or cannula) having a cutting edge 1730. The outer hypotube is located proximal to the inner member (and may therefore be referred to as a proximal hypotube), and may be coupled to the inner member so that it can be advanced once the inner member has engaged the ligamentum flavum. For example, the outer member (proximal hypotube) may be threaded so that it can be advanced by rotating, and screwed down over the ligamentum flavum, as shown in FIG. 17D. Thus, by clamping or compressing the outer and inner members, the portion of the ligamentum flavum between them may be cut and removed. In FIG. 17D, the ligamentum flavum access device, including the cut portion of the ligamentum flavum, may then be removed. In this example, a 5-20 mm portion of the ligamentum flavum may be removed in this fashion. Even after removal of the ligamentum flavum access device, access into the epidural space may be secured. For example, the device may not be removed until after a guide element (e.g., guidewire or the like) has been positioned through the ligamentum flavum. In some variations, only a portion of the epidural access device is removed. For example, the inner member may be removed, allowing access of other portions.

In still other variations, the opening through the ligamentum flavum may be expanded (e.g., FIGS. 14A-14G), and the expander may be left in while positioning a guidewire or the like. In any of the ligamentum flavum access devices described herein, a cannula, such as a tissue locking cannula, may be left in place for some time even after removal of all or a portion of the ligamentum flavum access device.

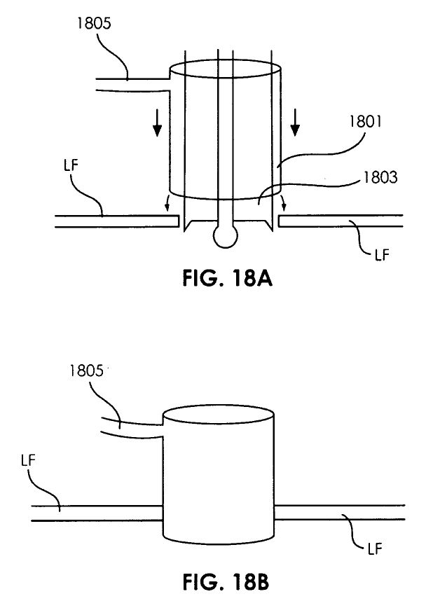

FIGS. 18A and 18B show one variation in which an access port, an anchoring cannula, is secured in the opening formed by the ligamentum flavum access device. For example, in FIG. 18A, after forming an opening through the ligamentum flavum, a cannula may be placed within the opening formed. The access cannula 1801 in this example is slid over the distal end of the ligamentum flavum access device 1803. Although in this example, the ligamentum flavum access device 1803 shown is a variation including a support member similar to the device shown in FIG. 17A-17D, any of the ligamentum flavum access devices described herein may be used.

The access cannula in this example thus spans the opening through the ligamentum flavum, and can be anchored in place using one or more anchors 1805. For example, the access cannula may include one or more barbs or members that either extend or are extendable outwards to engage tissue (including bone) and secure the cannula in place. As mentioned above, the access cannula may also be configured as a tissue locking cannula. In some variations, the distal end of the access cannula include one or more tissue-engaging surfaces.

An access cannula may also be referred to as a dilation tube 1801. In some variations the dilation tube is configured to further expand the opening formed by the ligamentum flavum access device. For example, the dilation tube may include walls configured to expand outwards to enlarge the opening. As shown in FIG. 18B, once the access tube/dilation cannula is in position to access the epidural space and span the ligamentum flavum (and may be anchored in place), the inner ligamentum flavum access device may be removed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

What is claimed is:

1. A method for accessing a spine of a patient, the method comprising:
advancing a cannula into the patient to contact a distal end of the cannula with spinal tissue including at least one of ligamentum flavum or vertebral periosteum;
removeably attaching the distal end of the cannula to at least one of the ligamentum flavum, periosteum and/or bone;
advancing a flexible guide member through the cannula and through at least one of the ligamentum flavum or vertebral periosteum to position a distal portion of the guide member in the epidural space of the spine; and
advancing the distal portion of the guide member at least partway into an intervertebral foramen of the spine.

2. The method of claim 1, further comprising forming an opening in the ligamentum flavum using a ligamentum flavum access tool within the cannula.

3. A method as in claim 1, wherein the cannula is advanced along with an epidural needle, with the cannula disposed over the needle as a sheath, the method further comprising removing the needle before advancing the guide member through the cannula.

4. A method as in claim 3, wherein removing the needle comprises:
ejecting the epidural needle proximally to remove a tip of the needle from the epidural space; and
sliding the needle proximally out of the cannula.

5. A method as in claim 1, wherein the cannula is advanced along with a blunt stylet, with the cannula disposed over the stylet as a sheath, the method further comprising removing the stylet before advancing the guide member through the cannula.

6. A method as in claim 1, wherein attaching the distal end of the cannula to the tissue comprises turning the cannula about its longitudinal axis in a first direction to couple one or more barbs disposed on its distal end with the tissue.

7. A method as in claim 6, further comprising turning the cannula about its longitudinal axis in a second direction, opposite the first direction, to release the cannula from the tissue, after advancing the guide member into the intervertebral foramen.

8. A method as in claim 1, further comprising, before advancing the guide member, advancing a rigid, blunt, cannulated probe through the cannula to position a distal end of the probe in the epidural space, wherein the guide member is advanced through the rigid probe.

9. A method as in claim 1, further comprising:
advancing a guidewire through the guide member to pass through the intervertebral foramen and out the patient's skin;
releasing the cannula from the spinal tissue; and
removing the cannula and the guide member from the patient, leaving the guidewire in place, extending into the patient, through the intervertebral foramen, and back out the patient.

10. A method as in claim 9, further comprising:
coupling a tissue removal device with the guidewire;
advancing the tissue removal device at least partway into the intervertebral foramen, using the guidewire; and
performing a tissue removal procedure in the patient's spine.

11. A method as in claim 1, further comprising transmitting stimulating current to at least one electrode disposed on the curved guide member to help determine a position of the guide member relative to nerve tissue.

12. A method as in claim 11, wherein transmitting the current comprises:
transmitting a first current to a first electrode disposed on an inner curvature surface of the guide member; and
transmitting a second current to a second electrode disposed on an outer curvature surface of the guide member.

13. A method as in claim 11, further comprising, before the transmitting step, advancing a sheath comprising at least one electrode over the guide member into the epidural space of the spine.

14. A method as in claim 1, further comprising, before advancing the guide member:
advancing at least one additional cannula over the attached cannula;
removeably attaching the additional cannula to the spinal tissue;
removing the cannula from the tissue; and
withdrawing the cannula through the additional cannula.

15. A method for accessing a spine of a patient, the method comprising:
advancing a cannula into the patient to contact a distal end of the cannula with spinal tissue including at least one of ligamentum flavum or vertebral periosteum;
removeably attaching the distal end of the cannula to at least one of the ligamentum flavum, periosteum and/or bone;
advancing a guide member through the cannula and through at least one of the ligamentum flavum or vertebral periosteum to position a distal portion of the guide member in the epidural space of the spine; and
advancing a guidewire through the guide member and at least partway into an intervertebral foramen of the spine.

16. A method for accessing an intervertebral foramen of a spine of a patient, the method comprising:
removeably attaching a distal end of a first tissue locking cannula to spinal tissue including at least one of ligamentum flavum or vertebral periosteum and/or bone;
passing at least a second tissue locking cannula over the first cannula;
removeably attaching a distal end of the second cannula to the spinal tissue;
removing the first cannula through the second cannula;
advancing a probe through the second cannula to position a distal portion of the probe in an epidural space of the patient's spine;
advancing a curved, at least partially flexible, cannulated guide member through the probe, such that when the distal portion exits the cannula it assumes a preformed curved shape; and
advancing the distal portion of the guide member at least partway into an intervertebral foramen of the spine.

17. A method as in claim 16, further comprising:
advancing a guidewire through the guide member to pass through the intervertebral foramen and out the patient's skin;
removing the probe from the patient;
releasing the second cannula from the spinal tissue; and
removing the cannula from the patient, leaving the guidewire in place, extending into the patient, through the intervertebral foramen, and back out the patient.

18. A method as in claim 17, further comprising, before advancing the probe:
passing at least a third tissue locking cannula over the second cannula;
removeably attaching a distal end of the third cannula to the spinal tissue; and
removing the second cannula through the third cannula.

19. A method as in claim 18, further comprising, before advancing the probe:
passing at least a fourth tissue locking cannula over the third cannula;
removeably attaching a distal end of the fourth cannula to the spinal tissue; and
removing the third cannula through the fourth cannula.

20. A system for accessing a spine of a patient, the system comprising:
at least one tissue locking cannula having multiple barbs disposed at one end for removeably attaching to spinal tissue including at least one of ligamentum flavum or vertebral periosteum and/or bone; and
a curved, at least partially flexible, cannulated guide member slideably passable through the cannula and having a distal portion configured to change from a straight shape within the cannula to a curved shape upon exiting the cannula, wherein the distal portion has a radius of curvature configured to position the distal portion at least partway into an intervertebral foramen of the spine when advanced through the cannula.

21. A system as in claim 20, wherein the barbs of the tissue locking cannula are configured so that they do not penetrate through the tissue.

22. A system as in claim 21, further comprising a rigid, cannulated probe slide ably passable through the cannula, wherein the curved guide member slide ably passes through the probe.

23. A system as in claim 22, wherein the guide member passes through an end aperture of the probe.

24. A system as in claim 22, wherein the guide member passes through a side aperture of the probe.

25. A system as in claim 22, further comprising at least one guidewire for passing through the guide member.

26. A system as in claim 25, further comprising a syringe for attaching to a proximal portion of the epidural needle.

27. A system as in claim 25, further comprising a tissue removal device removeably couplable with the guidewire for passing into the patient to remove spinal tissue.

28. A system as in claim 21, wherein the tissue locking cannula has an outer diameter of between 1 mm and 20 mm.

29. A system as in claim 28, wherein the barbs of the cannula face in one direction and attach to tissue by pressing the barbs against the tissue and turning the cannula along its longitudinal axis in a first direction.

30. A system as in claim 28, wherein the barbs release from tissue by turning the cannula along its longitudinal axis in a second direction opposite the first direction.

31. A system as in claim 21, wherein the guide member includes a rounded, atraumatic distal tip.

32. A system as in claim 21, wherein the at least one tissue locking cannula comprises multiple cannulas of different diameter, wherein a first cannula fits within a second cannula, and the second cannula fits within at least a third cannula.

33. A ligamentum flavum access tool device comprising:
an outer hypotube having a distal cutting edge; and
an inner member comprising an atraumatic tissue contacting region that is movable within the outer hypotube, and extends from the outer hypotube; wherein the inner member is configured to secure to a patient's ligamentum flavum; and
a sensor to detect entry into the epidural space.

34. The device of claim 33, wherein the sensor comprises a loss of resistance detector configured to determine when the inner member is within the epidural space.

35. The device of claim 33, wherein the inner member comprises a vacuum port configured to provide a vacuum for securing the inner member to the ligamentum flavum.

36. The device of claim 33, further comprising at least one support element extendable from the inner member when the inner member is within the epidural space.

37. The device of claim 33, wherein the atraumatic tissue contacting region of the inner member includes a distal head and a proximal neck that has a smaller diameter than the distal head, wherein the ligamentum flavum may be secured around the proximal neck after the distal head has penetrated the ligamentum flavum.

38. The device of claim 33, further comprising a threaded region on an outer surface of the device that is configured to mate with a cannula so that the device may be controllably advanced within the cannula by rotation.

39. The device of claim 33, further comprising an internal threaded region in communication with the inner atraumatic tissue contacting member so that it may be moved relative to the outer hypotube.

40. A ligamentum flavum access tool device comprising:
an elongate body;
a distal tip member comprising an atraumatic tissue contacting region configured as a leading head;
a cutting surface that is located proximal to the distal tip member; and
a loss of resistance detector, configured to determine when the distal tip member is within the epidural space.

41. The device of claim 40, wherein the cutting surface is located on a proximal side of the leading head of the distal tip member.

42. The device of claim 40, wherein the cutting surface is a cutting edge of a hypotube in which the distal tip member may axially move.

43. The device of claim 40, further comprising at least one support element extendable from the distal tip member when the distal tip member is within the epidural space.

44. The device of claim 40, wherein the distal tip member is axially movable relative to the cutting surface.

45. The device of claim 40, further comprising a threaded region on an outer surface of the device that is configured to mate with a cannula so that the device may be controllably advanced within the cannula by rotation.

46. The device of claim 40, further comprising an internal threaded region in communication with the distal tip member so that the distal tip member may be moved relative to the cutting surface.

47. A method of accessing the spine of a patient comprising:
anchoring the distal end of a cannula in contract with a patient's ligamentum flavum;
advancing a ligamentum flavum access tool within the cannula in a controlled manner;
penetrating the ligamentum flavum with the ligamentum flavum access tool to access the epidural space; and
forming an opening in the ligamentum flavum with the ligamentum flavum access tool.

48. A method of accessing the spine of a patient comprising:
anchoring the distal end of a cannula in contract with the patient's ligamentum flavum;
advancing a ligamentum flavum access tool distally within the cannula in a controlled manner, wherein the ligamentum flavum access tool comprises
an outer hypotube having a distal cutting edge, and
an inner member comprising an atraumatic tissue contacting region that is movable within the outer hypotube, and extends distally from the outer hypotube;
securing the ligamentum flavum to the atraumatic tissue contacting region of the ligamentum flavum access tool; and
cutting an opening in the ligamentum flavum with the cutting edge of the proximal hypotube.

49. The method of claim 48, wherein the step of securing the ligamentum flavum to the atraumatic tissue contacting region of the ligamentum flavum access tool comprises deploying one or more support elements from the atraumatic tissue contacting region when atraumatic tissue contacting region is within the epidural space.

50. The method of claim 48, wherein the step of securing the ligamentum flavum to the atraumatic tissue contacting region of the ligamentum flavum access tool comprises penetrating the ligamentum flavum with the atraumatic tissue contacting region until the atraumatic tissue contacting region is within the epidural space as determined by the loss of resistance detector.

51. The method of claim 48, wherein the step of cutting an opening in the ligamentum flavum comprises moving the atraumatic tissue contacting region secured to the ligamentum flavum proximally so that the ligamentum flavum is cut by the cutting edge of the outer hypotube.

52. The method of claim 48, wherein the step of cutting an opening in the ligamentum flavum comprises moving the cutting edge of the outer hypotube distally relative to the atraumatic tissue contacting region secured to the ligamentum flavum.

53. The method of claim 48, further comprising removing the ligamentum flavum access tool from the cannula.

54. The method of claim 48, wherein the step of anchoring the distal end of the cannula comprises removeably attaching the distal end of the cannula to the ligamentum flavum.

55. The method of claim 48, wherein the step of anchoring the distal end of the cannula comprises anchoring the cannula to a surgical access platform.

56. The method of claim 48, wherein the step of advancing the ligamentum flavum access tool comprises rotating the tool relative to the cannula to advance the tool along a threaded region.

57. A method of accessing the spine of a patient comprising:
anchoring the distal end of a cannula in contract with the ligamentum flavum;
advancing a ligamentum flavum access tool distally within the cannula in a controlled manner, wherein the ligamentum flavum access tool comprises
a proximal cutting surface,
a distal tip member comprising an atraumatic tissue contacting region configured as a leading head, and
a loss of resistance detector;
penetrating the ligamentum flavum with the atraumatic leading head of the tip region until the atraumatic leading head accesses the epidural space as determined by the loss of resistance detector;
cutting the ligamentum flavum with the proximal cutting surface; and
removing the ligamentum flavum access tool from the cannula.

58. The method of claim 57, wherein the step of anchoring the distal end of the cannula comprises removeably attaching the distal end of the cannula to the ligamentum flavum.

59. The method of claim 57, wherein the step of anchoring the distal end of the cannula comprises anchoring the cannula to a surgical access platform.

60. The method of claim 57, wherein the step of advancing the ligamentum flavum access tool comprises rotating the tool relative to the cannula to advance the tool along a threaded region.

61. The method of claim 57, wherein the step of cutting the ligamentum flavum with the proximal cutting surface comprises compressing the ligamentum flavum between the distal tip member and the proximal cutting surface.

62. The method of claim 57, wherein the step of cutting the ligamentum flavum with the proximal cutting surface comprises retracting the distal tip member so that the proximal cutting surface can engage the ligamentum flavum.

63. The method of claim 57, further comprising deploying one or more support elements from the distal tip member when the distal tip member is within the epidural space.

64. A method of accessing the spine of a patient comprising:
anchoring the distal end of a cannula in contract with the ligamentum flavum;
advancing a ligamentum flavum access tool distally within the cannula in a controlled manner, wherein the ligamentum flavum access tool comprises
a proximal hypotube having an expandable distal end, and
a distal tip member comprising an atraumatic leading head;
penetrating the ligamentum flavum with the atraumatic leading head of the tip region until the expandable distal end of the hypotube spans the ligamentum flavum; and
dilating the expandable distal end of the hypotube to expand an opening in the ligamentum flavum.

65. The method of claim 64, wherein the step of dilating the expandable distal end of the hypotube comprises withdrawing the distal tip member proximally through the hypotube to expand the distal end of the hypotube.

66. The method of claim 64, further comprising removing the ligamentum flavum access tool from the cannula.

67. The method of claim 64, further comprising removing the atraumatic leading head from the hypotube to allow access to the patient's epidural space through the cannula.

68. The method of claim 64, wherein the step of penetrating the ligamentum flavum comprises determining when the distal end of the hypotube has entered the epidural space.

69. The method of claim 64, wherein the step of anchoring the distal end of the cannula comprises removeably attaching the distal end of the cannula to the ligamentum flavum.

70. The method of claim 64, wherein the step of anchoring the distal end of the cannula comprises anchoring the cannula to a surgical access platform.

71. The method of claim 64, wherein the step of advancing the ligamentum flavum access tool comprises rotating the tool relative to the cannula to advance the tool along a threaded region.
